SEQUENCE LISTING (37 sequences)

SEQ ID NO: 1 - DNA, 17 bp, Artificial
  LR0R primer
acccgctgaacttaagc

SEQ ID NO: 2 - DNA, 17 bp, Artificial
  LR6 primer
cgccagttctgcttacc

SEQ ID NO: 3 - DNA, 17 bp, Artificial
  LR3R primer
gtcttgaaacacggacc

SEQ ID NO: 4 - DNA, 17 bp, Artificial
  LR3 primer
ccgtgtttcaagacggg

SEQ ID NO: 5 - DNA, 19 bp, Artificial
  ITS1 primer
tccgtaggtgaacctgcgg

SEQ ID NO: 6 - DNA, 20 bp, Artificial
  ITS4 primer
tcctccgcttattgatatgc

SEQ ID NO: 7 - DNA, 1052 bp, Myrothecium
  misc_feature: 28s rDNA sequence of the strain MUCL39210
cagggattgcctcagtaacggcgagtgaagcggcaacagctcaaatttgaaatctggccccccggcccgagttgtaatttgcagaggatgcttttggcgaggtgccttccgagttccctggaacgggacgccatagagggtgagagccccgtctggtcggacaccgagcctctgtaaagctccttcgacgagtcgagtagtttgggaatgctgctcaaaatgggaggtatatgtcttctaaagctaaatattggccagagaccgatagcgcacaagtagagtgatcgaaagatgaaaagcactttgaaaagagagttaaacagcacgtgaaattgttaaaagggaagcgtttatgaccagacttggcccggttgatcatccagcgttctcgctggtgcactctgccggtccaggccagcatcagttcgtcgcgggggataaaggtttcgggaatgtggctcctccgggagtgttatagcccgttgcgtaataccctgcggtggactgaggtccgcgcttctgcaaggatgctggcgtaatggtcatcaacgacccgtcttgaaacacggaccaaggagtcgtcttcgtatgcgagtgttcgggtgtaaaacccctacgcgtaatgaaagtgaacgcaggtgagagcttcggcgcatcatcgaccgatcctgatgttctcggatggatttgagtaagagcatacggggccggacccgaaagaaggtgaactatgcctgtatagggtgaagccagaggaaactctggtggaggctcgcagcggttctgacgtgcaaatcgatcgtcaaatatgggcatgggggcgaaagactaatcgaaccttctagtagctggtttccgccgaagtttccctcaggatagcagtgttgaactcagttttatgaggtaaagcgaatgattagggactcgggggcgctatttagccttcatccattctcaaactttaaatatgtaagaagcccttgttacttagttgaacgtgggcattcgaatgtatcaacactagtgggccatttttggtaagcagaactggc

SEQ ID NO: 8 - DNA, 1040 bp, Myrothecium
  misc_feature: 28s rDNA sequence of the strain MUCL11831
cagggattgccttagtaacggcgagtgaagcggcaacagctcaaatttgaaatctggacctagtcccgagttgtaatttgcagaggatgattttggcgcggtgccttccaagttccctgggacgggacgccggagagggtgagagccccgtcaggttggacaccaagcctatgtaaatctccttcgacgagtcgagtagtttgggaatgctgctctaaatgggaggtatatgtcttctaaagctaaataccggccagagaccgatagcgcacaagtagagtgatcgaaagatgaaaagcactttggaaagagagttaaacagcacgtgaaattgttaaaagggaagcgtttacgaccagacttgtgccggttgatcatccagcgttctcgctggtgcactctgccggcccaggccagcatcagttcgccgcgggggataaaggcgtcgggaatgtggctcccccgggagtgttatagcccttcgcgcaataccctgaggcggactgaggttcgcgcattcgcaaggatgctggcgtaatggtcgtcaacgacccgtcttgaaacacggaccaaggagtcgtcttcgtatgcgagtgttcgggtgtaaaacccctacgcgtaatgaaagtgaacgcaggtgagagcttcggcgcatcatcgaccgatcctgatgttctcggatggatttgagtaagagcatacggggccggacccgaaagaaggtgaactatgcctgtatagggtgaagccagaggaaactctggtggaggctcgcagcggttctgacgtgcaaatcgatcgtcaaatatgggcatgggggcgaaagactaatcgaaccttctagtagctggtttccgccgaagtttccctcaggatagcagtgttgaactcagttttatgaggtaaagcgaatgattagggactcgggggcgctatttagccttcatccattctcaaactttaaatatgtaagaagcccttgttgcttaattgaacgtgggcattcgaatgtatcaacactagtgggccatttttggtaa

SEQ ID NO: 9 - DNA, 1012 bp, Myrothecium
  misc_feature: 28s rDNA sequence of the strain CBS449.71
cagggattgcctcagtaacggcgagtgaagcggcaacagctcaaatttgaaatctggccctaggcccgagttgtaatttgcagaggatgcttttggcaaggtgccttccgagttccctggaacgggacgccatagagggtgagagccccgtctggtcggacaccgagcctctgtaaagctccttcgacgagtcgagtagtttgggaatgctgctcaaaatgggaggtatatgtcttctaaagctaaataccggccagagaccgatagcgcacaagtagagtgatcgaaagatgaaaagcactttgaaaagagagttaaatagcacgtgaaattgttgaaagggaagcgtttatgaccagacttggcccggttgatcatccagccttctggctggtgcactctgccggtccaggccagcatcagttcgtcgcgggggataaaggtttcgggaatgtagctcctccgggagtgttatagcccgttgcgtaataccctgcggtggactgaggtccgcgctctgcaaggatgctggcgtaatggtcatcaacgacccgtcttgaaacacggaccaaggagtcgtcttcgtatgcgagtgttcgggtgtaaaacccctacgcgtaatgaaagtgaacgcaggtgagagcttcggcgcatcatcgaccgatcctgatgttctcggatggatttgagtaagagcatacggggccggacccgaaagaaggtgaactatgcctgtatagggtgaagccagaggaaactctggtggaggctcgcagcggttctgacgtgcaaatcgatcgtcaaatatgggcatgggggcgaaagactaatcgaaccttctagtagctggtttccgccgaagtttccctcaggatagcagtgttgaactcagttttatgaggtaaagcgaatgattagggactcgggggcgctatttagccttcatccattctcaaactttaaatatgtaagaagcccttgttgcttaattgaacgtgggcattcgaatgta

SEQ ID NO: 10 - DNA, 1038 bp, Myrothecium
  misc_feature: 28s rDNA sequence of the strain IMI140595
cagggattgcctcagtaacggcgagtgaagcggcaacagctcaaatttgaaatctggccctaggcccgagttgtaatttgcagaggatgcttttggcgaggtgccttccgagttccctggaacgggacgccatagagggtgagagccccgtctggtcggacaccgagcctctgtaaagctccttcgacgagtcgagtagtttgggaatgctgctcaaaatgggaggtatatgtcttctaaagctaaataccggccagagaccgatagcgcacaagtagagtgatcgaaagatgaaaagcactttgaaaagagagttaaacagcacgtgaaattgttgaaagggaagcgtttatgaccagacttggcccggttgatcatccagcgttctcgctggtgcactctgccggtccaggccagcatcagttcgccgcgggggataaaggtttcgggaatgtggctcctccgggagtgttatagcccgttgcgtaataccctgcggtggactgaggtccgcgcttctgcaaggatgctggcgtaatggtcatcaacgacccgtcttgaaacacggaccaaggagtcgtcttcgtatgcgagtgttcgggtgtaaaacccctacgcgtaatgaaagtgaacgcaggtgagagcttcggcgcatcatcgaccgatcctgatgttctcggatggatttgagtaagagcatacggggccggacccgaaagaaggtgaactatgcctgtatagggtgaagccagaggaaactctggtggaggctcgcagcggttctgacgtgcaaatcgatcgtcaaatatgggcatgggggcgaaagactaatcgaaccttctagtagctggtttccgccgaagtttccctcaggatagcagtgttgaactcagttttatgaggtaaagcgaatgattagggactcgggggcgctatttagccttcatccattctcaaactttaaatatgtaagaagcccttgttacttagttgaacgtgggcattcgaatgtatcaacactagtgggccatttttggt

SEQ ID NO: 11 - DNA, 1044 bp, Myrothecium
  misc_feature: 28s rDNA sequence of the strain IMI29040
cagggattgcctcagtaacggcgagtgaagcggcaacagctcaaatttgaaatctggccccccggcccgagttgtaatttgcagaggatgcttttggcaaggtgccttccgagttccctggaacgggacgccatagagggtgagagccccgtctggtcggacaccgagcctctgtaaagctccttcgacgagtcgagtagtttgggaatgctgctcaaaatgggaggtatatgtcttctaaagctaaataccggccagagaccgatagcgcacaagtagagtgatcgaaagatgaaaagcactttgaaaagagagttaaatagcacgtgaaattgttgaaagggaagcgtttatgaccagacttggcccggttgatcatccagcgttctcgctggtgcactctgccggtccaggccagcatcagttcgtcgcgggggataaaggcttcgggaatgtagctctcttcggggagtgttatagcccgttgtgtaataccctgcggtggactgaggtccgcgctctgcaaggatgctggcgtaatggtcatcacgaccgtcttgaaacacggaccaaggagtcgtcttcgtatgcgagtgttcgggtgtaaaacccctacgcgtaatgaaagtgaacgcaggtgagagcttcggcgcatcatcgaccgatcctgatgttctcggatggatttgagtaagagcatacggggccggacccgaaagaaggtgaactatgcctgtatagggtgaagccagaggaaactctggtggaggctcgcagcggttctgacgtgcaaatcgatcgtcaaatatgggcatgggggcgaaagactaatcgaaccttctagtagctggtttccgccgaagtttccctcaggatagcagtgttgaactcagttttatgaggtaaagcgaatgattagggactcgggggcgctatttagccttcatccattctcaaactttaaatatgtaagaagcccttgttgcttaattgaacgtgggcattcgaatgtatcaacactagtgggccatttttggtaagcag

SEQ ID NO: 12 - DNA, 445 bp, Myrothecium
  misc_feature: ITS sequence of the strain MUCL39210
gcgggaccgccccggcgccctcgcggcccggacccaggcgcccgccggagaccccaaactctatgttttactgtacatctcctctgagtgacacataaacaataaataaaaacttttaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgttcgagcgtcatttcaaccctcaggcccccagtgcctggcgttggggatcggcaccagggcgtccgcgcaagcggtctccccgccggccccgaaatctagtggcggtctcgctgtagtcctcctctgcgtagtagcacaacctcgcagctggaactcggcggtggccctgccgttaaacaccccacttctga

SEQ ID NO: 13 - DNA, 459 bp, Myrothecium
  misc_feature: ITS sequence of the strain MUCL11831
gcgggctcagcccccgcgccctcgccggcgccgggaaacaggcgcccgccggagacccaaactcaatgtttttcatgcagtattatctgagtggcaaacgcaaaaaataaatcaaaacttttaacaacggatctcttggctctggcatcgatgaagaacgcagcgaaatgcgataagtaatgtgaattgcagaattcagtgaatcatcgaatctttgaacgcacattgcgcccgcgagtattctcgcgggcatgcctgtccgagcgtcatttcaaccctcagggcccgcctgcttgcaggcgcggcgctggtgttggggatcggccctaaaccgccgtcccccaaattcagtggcggtctcgctgcagcctcccctgcgtagtagcaacactcgcatgcggagcgcggcgcggccacgccgtaaaacccccgactttctgaacgttgacctcggatcag

SEQ ID NO: 14 - DNA, 423 bp, Myrothecium
  misc_feature: ITS sequence of the strain CBS449.71
gcgggaccgccccggcgccttcgggcccggaaccaggcgcccgccggaggccccaaactcttatgtctttagtggttttctcctctgagtgacacataaacaaataaataaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgttcgagcgtcatttcaaccctcaggcccccagtgcctggtgttggggatcggcccagccttctcgcaaggccgccggccccgaaatctagtggcggtctcgctgtagtcctcctctgcgtagtagcacaacctcgcagttggaacgcggcggtggccatgccgttaaa

SEQ ID NO: 15 - DNA, 445 bp, Myrothecium
  misc_feature: ITS sequence of the strain IMI140595
gcgggaccgccccggcgccctcgcggcccggacccaggcgcccgccggagaccccaaactctatgttttactgtacatctcctctgagtgacacataaacaataaataaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgttcgagcgtcatttcaaccctcaggcccccagtgcctggcgttggggatcggctcaggggcgaccgcgcaagcggccgcttcccgccggccccgaaatctagtggcggtctcgctgtagtcctcctctgcgtagtagcacaacctcgcagctggaacgcggcggtggccctgccgtaaaacaccccacttct

SEQ ID NO: 16 - DNA, 434 bp, Myrothecium
  misc_feature: ITS sequence of the strain IMI290405
gcgggaccgccccggcgccctcgggcccggaaccaggcgcccgccggaggccccaaaccctcatgtctttagtggttttctcctctgagtgacacataaacaaataaataaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgttcgagcgtcatttcaaccctcaggcccccagtgcctggcgttggggatcggcagcagggcgtcaagcccgccggccccgaaatctagtggcggtctcgctgtagtcctcctctgcgtagtagcacaacctcgcagttggaacgcggcggtggccatgccgttaaacaccccacttctg

SEQ ID NO: 17 - DNA, 24 bp, Artificial
  AMY1 primer
ggaattccacagaaggcatttatg

SEQ ID NO: 18 - DNA, 23 bp, Artificial
  AMY2 primer
gctctagagcaaccaccaggtca

SEQ ID NO: 19 - DNA, 17 bp, Artificial
  Gpd1 primer
ggnatcaayggnttcgg

SEQ ID NO: 20 - DNA, 20 bp, Artificial
  Gpd2 primer
gtgswgswggggatgatgtt

SEQ ID NO: 21 - DNA, 23 bp, Artificial
  Gpd3 primer
ggtcgtatcgtnttycgnaaygc

SEQ ID NO: 22 - DNA, 23 bp, Artificial
  Gpd4 primer
ggagccaggcagttggtngtrca

SEQ ID NO: 23 - DNA, 836 bp, Myrothecium
  misc_feature: gpd gene sequence of the strain MUCL11831
gtggtggtggggatgatgttctgggcagcgccacggccaccgcgccagtccttggcggagggaccgtcgacggtcttctgggtggcagtgtaggagtggacggtggtcatgagaccctcaatgatggtgtacttgtcgttgaggaccttggcgaggggagccaggcagttggtggtgcacgaggcgttggagatgacatcggcgctgccgtcgtaggtcttctcgttgacgcccatcacgtacatgggggcatcggcggagggggcggagatgatgaccttcttggcaccgccagcaaggtgagccttggccttgtcggtggtggtgaagacaccggtggactcgacaatgtactcggcgccggtctccttccaggggatggaggcggggtcacgctcggtgtagaagcggaccttcttgccgttgacgctcaggtcagcgccatcggcctcgacctcacccttgaagagaccgtgggtggagtcatacttgagcatgtaggcctagatggtggtcagctaaagcgctcatttcaagacaaagaaagcagatgtcaaggttggcgggaaaagacgatggaggggcacgggtttggacatggttgcaggtaggtggggtgcaacggcccatgtcattgcaagcatgccatgtcgggttttgcccctcggattggatttcttttttcgcgccgcatcatgtaaagtggggggaggggcagcactcacggcgtacttggtctcgatgaaggggtcgttgacggcgacaatctcaatgtcggggtgctcgacggcgttgcggaagacgatacgaccgatgcggccgaacccattgatccc

SEQ ID NO: 24 - DNA, 954 bp, Myrothecium
  misc_feature: gpd gene sequence of the strain CBS449.71
gtgcagctggggatgatgttctgggcagcaccacggccaccgcgccagtccttggcggagggaccgtcaacggtcttctgggtggcagtgtagagtggacggtggtcatgagaccctcaatgatggtgaacttgtcgtggatgaccttggcgaggggagccaggcagttggtggtgcaagaagcgttggagatgacgtcggcgctgccgtcgtaggtctcctcgttgacacccataacgtacatgggggcatcggcggagggagcagagatgatgaccttcttggcaccacccttcaagtgagcagcagccttgtccttggtggtgaagacaccagtggactcgacgatgtactcggcgccagtctccttccaggggatggcagcggggtcgcgctcagtgtagaagcggaccttcttgccgttgacagtgaggtcggcaccatcgacggagacctcacccttgaagagaccgtgggtagagtcatacttgagcatgtaggcctagtgaacaggggtggttagcggaatggccggcagagagagagtaattgcggcatgacgaggcgtgggagggaggagcagtccctcgccatgacgatagcattggctattgattcattcgccgccttgacagagggctcgttgaactgcaccaacgcatgatatcatcatggaggggcaaatattgacgtgtaatggtggggtgaatggcagagtggttgcgttttttctgcccctcacttggagatggccgtgtcgcgtctgcccctcgtctccctgcaagtgcatggtggactgcagctcgcttgcctagttgttggctgaagggaaacagcacttacagcgtacttgggctcaatgaagggatcgttgacggcaacgatctcgacgtcgtcgtgctcgacggcgttgcggaagacgatacgaccaatgcggccgaacccattgatccc

SEQ ID NO: 25 - DNA, 1055 bp, Myrothecium
  misc_feature: gpd gene sequence of the strain IMI290405
gtggtggaggggatgatgttctgggcagcaccacggccaccacgccagtccttggcggagggaccatcgacggtcttctgggtggcagtgtaggagtggacagtggtcatgagaccctcaatgatggtgaacttgtcgtgaatgaccttggcgagaggagccaggcagttggtggtgcaggaggcgttggagatgacgtcggcgctgccgtcgtaggtctcctcgttgacacccataacgtacatgggagcatcagcagagggggcagagatgatgaccttcttggcaccacccttcaagtgagcagcggccttgtccttggtggtgaagacaccggtggactcgacgatgtagtcggcgccagtctccttccaagggatggcagcggggtcacgctcagtgtagaagcggaccttcttgccgttgacggtcaggtcagcgccatcgacggtgacctcacccttgaagacgccgtgggtggagtcatacttgagcatgtaggcctatgcgtggatggtggtgggaagcatgagtgaattggagggattgcgtgagggtgatgaagcatcattgtggtgtgtcaatggggctgtttctgctgctgctggcggcggttgttggtggtggtgacaaaagaaatttgttgagcgggaaagggatagacggcggcgcatgatatcatggaggggcaaatattgacgcgctgatgatagtggggtgatttttggaggcacctggttttgtctttggttgcattttttctgcccctcactcggtcggtccgtgtctgcggcgcgcgtctgcccctcctctgtctgcacagagtgcatgctgggctgcagccagctccgttgcccgctcgctcgctcgcctgcgtgccttgtccctttggagctgaggggaaagaggtgggatcgagatcacaatcaaaaggttgtactcacagcgtaggtgggctcaatgaagggatcgttgacggcaacgatctcgacgtcggagtgctcgacggcgttgcggaagacgatacgaccaatgcggccgaacccattgatacc

SEQ ID NO: 26 - DNA, 850 bp, Myrothecium
  misc_feature: gpd gene sequence of Myrothecium gramineum (Xepiculopis graminea) MUCL39210
gccgtcgagcactccgacgtcgagatcgttgccgtcaacgaccccttcattgagcccaagtacgctgtaagtgctgcttctgcttcccctcagtcgacgagcgagcccaaagccgagctgcagctagcggagccatgcgctgcctgcatgccactgcataacagcagctagaggaggggtacacggccgcgcgcgcagacacacatacaacaccaccaccaccaaaaggaggggcagaaaaaatccagcattgtccgatttcaccccaccatctcacgtcaaccaatttgcccctccatgatatcatgtgtccgcgcccagctcaacacgtccacctcctctggccaatggcgagcgcattgatgctttgatgagcggaaacgacgctgaggccctcagcctcgtcgtcgctgccgctgccgccgcgcgccgctcacgcatcggcgggctcccgtcgctgggcttcaattgacatgacatgatgcatggccaccgtgctaaccacccctgtgtctgtccgataggcctacatgctcaagtatgactctacccacggtctcttcaagggtgaggtcaccgtcgatggcgatgacctgaccgtcaacggcaagaaggtccgcttctacactgagcgtgaccccgccgccatcccctggaaggagactggtgccgagtacattgtcgagtccaccggtgtcttcaccaccaaggacaaggctgctgctcacctgaagggtggtgccaagaaggtcatcatctctgccccctctgccgatgcccccatgtacgttatgggtgtcaacgaggagacctacgacggcagcgccgacgtcatctccaacgcttcttg

SEQ ID NO: 27 - protein, 130 aa, Myrothecium
  MISC_FEATURE: glyceraldehyde 3-P dehydrogenase sequence of Myrothecium gramineum (Xepiculopis graminea) MUCL39210
AVEHSDVEIVAVNDPFIEPKYAAYMLKYDSTHGLFKGEVTVDGDDLTVNGKKVRFYTERDPAAIPWKETGAEYIVESTGVFTTKDKAAAHLKGGAKKVIISAPSADAPMYVMGVNEETYDGSADVISNAS

SEQ ID NO: 28 - DNA, 21 bp, Artificial
  hphpCSN431 primer
atgcctgaactcaccgcgacg

SEQ ID NO: 29 - DNA, 18 bp, Artificial
  hphpCSN432 primer
ctattcctttgccctcgg

SEQ ID NO: 30 - DNA, 16 bp, Artificial
  GPD2 primer
tctggcatgcggagag

SEQ ID NO: 31 - DNA, 16 bp, Artificial
  AMY5 primer
cgatgatgccctgcca

SEQ ID NO: 32 - DNA, 850 bp, Myrothecium; identical to SEQ ID NO: 26
  CDS (1)..(66)
  misc_feature (1)..(850): partial nucleotide sequence and its deduced protein sequence of the Myrothecium gramineum MUCL39210 glyceraldehyde-3-P dehydrogenase gene

SEQ ID NO: 33 - protein, 22 aa, Myrothecium
AVEHSDVEIVAVNDPFIEPKYA

SEQ ID NO: 34 - protein, 108 aa, Myrothecium
AYMLKYDSTHGLFKGEVTVDGDDLTVNGKKVRFYTERDPAAIPWKETGAEYIVESTGVFTTKDKAAAHLKGGAKKVIISAPSADAPMYVMGVNEETYDGSADVISNAS

SEQ ID NO: 35 - protein, 204 aa, Myrothecium
  MISC_FEATURE: partial GPD protein sequence from the strain MUCL39210
GINGFGRIGRIVFRNAVEHPDIEIVAVNDPFIETKYAAYMLKYDSTHGLFKGEVEADGADLSVNGKKVRFYTERDPASIPWKETGAEYIVESTGVFTTTDKAKAHLAGGAKKVIISAPSADAPMYVMGVNEKTYDGSADVISNASCTTNCLAPLAKVLNDKYTIIEGLMTTVHSYTATQKTVDGPSAKDWRGGRGAAQNIIPTT

SEQ ID NO: 36 - protein, 204 aa, Myrothecium
  MISC_FEATURE: partial GPD protein sequence from the strain MUCL11831
GINGFGRIGRIVFRNAVEHDDVEIVAVNDPFIEPKYAAYMLKYDSTHGLFKGEVSVDGADLTVNGKKVRFYTERDPAAIPWKETGAEYIVESTGVFTTKDKAAAHLKGGAKKVIISAPSADAPMYVMGVNEETYDGSADVISNASCTTNCLAPLAKVIHDKFTIIEGLMTTVHSTLPPRRPLTVPPPRTGAVAVVLPRTSSPAA

SEQ ID NO: 37 - protein, 204 aa, Myrothecium
  MISC_FEATURE: partial GPD protein sequence from the strain CBS449.71
GINGFGRIGRIVFRNAVEHSDVEIVAVNDPFIEPTYAAYMLKYDSTHGVFKGEVTVDGADLTVNGKKVRFYTERDPAAIPWKETGADYIVESTGVFTTKDKAAAHLKGGAKKVIISAPSADAPMYVMGVNEETYDGSADVISNASCTTNCLAPLAKVIHDKFTIIEGLMTTVHSYTATQKTVDGPSAKDWRGGRGAAQNIIPST

(19) United States
(12) Patent Application Publication
(10) Pub. No.: US 2006/0240509 A1
(43) Pub. Date: Oct. 26, 2006

(54) Myrothecium sp transformation and expression system

(76) Inventors: Jean-Luc Jonniaux, Tienen (BE); Emmanuel Valepyn, Gilly (BE); Annie-Marie Corbisier, Louvain-La-Neuve (BE); Thierry Dauvrin, Couthuin (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/525,984

(22) PCT Filed: Aug. 29, 2003
(86) PCT No.: PCT/BE03/00143

§ 371 Date: Sep. 16, 2005

Related U.S. Application Data
(60) Provisional application No. 60/407,843, filed on Aug. 30, 2002.

(51) Int. Cl. C12P 21/06; C12N 1/16; C12N 9/00; C12N 9/32; C12N 9/42; C12N 15/74

(52) U.S. Cl. 435/69.1; 435/254.1; 435/484; 435/183; 435/209; 435/204

(57) ABSTRACT

The present invention is related to a transformation and an expression system in which Myrothecium sp. host cells are used to express homologous or heterologous proteins or are used to genetically engineer metabolic pathways.

### FIELD OF THE INVENTION

The present invention is related to a method to express proteins or to modify protein expression in a microbial host.

### BACKGROUND OF THE INVENTION

Since the advent of the genetic engineering a number of living cells has been transformed with DNA in order to produce homologous or heterologous proteins or to modify their metabolism by introducing for example new metabolic pathways. The variety of cloning hosts spans from bacteria such as Escherichia coli till human cells and comprises organisms as diverse as yeasts, fungi or insect cells.

Among these organisms, the filamentous fungi have been the subjects of many studies. Indeed some of these organisms are industrially important, such as Aspergillus sp., Trichoderma sp. or Fusarium sp.

The first report of a DNA-mediated transformation of a filamentous fungi was published in 1973 using Neurospora crassa (Mishra, N. C. and Tatum E. L., 1973. Non-Mendelian inheritance of DNA-induced inositol independence in Neurospora. Proc. Nat. Acad. Sci. USA. 70:3875-3879). Later on, a protoplast transformation system has been developed for the same organism (Case et al, 1979. Efficient transformation of Neurospora crassa by utilising hybrid plasmid DNA. Proc. Nat. Acad. Sci. USA. 76:5259-5263). Transformation of Podospora anserina (Stahl et al, 1982. Replication and expression of a bacterial-mitochondrial hybrid plasmid in the fungus Podospora anserina. Proc. Nat. Acad. Sci. USA. 79:3641-3645) and Aspergillus nidulans (Tilburn et al, 1983. Transformation by integration in Aspergillus nidulans. Gene. 26:205-221; Ballance et al, 1983. Transformation of Aspergillus nidulans by the orotidine-5′-phosphate decarboxylase gene of Neurospora crassa. Biochem. Biophys. Res. Comm. 112:284-289) were also reported. The same techniques have been successfully used to transform the commercially important industrial fungi Aspergillus niger (Buxton et al, 1985. Transformation of Aspergillus niger using the argB gene of Aspergillus nidulans. Gene. 37:207-214), Aspergillus oryzae (Gomi et al, 1987. Agric. Biol. Chem. 51:2549-2555) and Trichoderma reesei (Pentillä et al, 1988.Gene. 61:155-164).

Today transformations systems have been reported for many fungal classes (see for example the chapter on Genetic manipulation of fungi by transformation by Lemke P. A. and Peng M. in The Mycota volume II Genetics and Biotechnology, Kück ed, Springer-Verlag, 1995).

Some of these are patented (see for example patent EP0184438B1 on “Transforming Aspergillus and plasmids for use therein” or patent application WO9602653 on “Thermophilic fungal expression system”). Also are described parts of transformation systems such as the use of promoters or plasmids (For example EP0489718 “Process for the production of protein products in Aspergillus oryzae and a promoter for use in Aspergillus”).

Enzymes are proteins that are produced by all living organisms. They are highly specific biological catalysts that speed up chemical reactions selectively.

They are produced industrially for a number of application ranging from detergent formulation to food application (baking, fruit processing, . . . ) (for reviews see the books of Uhlig, H., Industrial enzymes and their applications, 1998, John Wiley and sons, Inc; Enzymes in food technology, 2002, Whitehurst, R. J. and Law, B. A. eds, Sheffield Academic Press; Industrial enzymology, 1996, Godfrey, T. and West, S. eds., Macmillan Press ltd).

About 90% of all enzymes used in industrial processing are produced by fermentation of microorganisms.

Today very few species of microorganisms are used for industrial enzyme production. This is a limitation imposed by producing companies needing the widest market range for their products, including food processing.

However, this situation is changing as new opportunities outside food processing are appearing. Also the increasing introduction of productions using genetically manipulated microorganisms strengthens the need to find new organisms to be used. The genetic engineering allows also the possibility to produce enzymes naturally produced by microorganims having poor growing properties in fermentors.

Besides enzyme production, genetically modified microorganisms are used for many other applications such as but not restricted to therapeutical drugs production (antibiotics, anticancer, . . . ), new metabolic pathway expression, . . . .

Therefore, there is still a need for expression systems that would allow high or modulated levels of production of various compounds.

The diversity of the microbial world is today largely underexploited. It has been estimated that less than 1% of the microorganisms living on earth have already been discovered. Among those identified, very few have been the subject of investigations other than morphological or taxonomical ones.

It is therefore the subject of the present invention to present the results of a screening of this microbial diversity and to characterise a microbial genus that would present the characteristics required for a performing cloning host cell: good growth in industrial conditions, transformability, high expression level or well modulated expression level of homologous or heterologous genes or gene sets.

### SUMMARY OF THE INVENTION

The present invention relates to an isolated Myrothecium host cell comprising at least one recombinant DNA construct, or nucleic acid construct, for the modulated expression of homologous genes and/or for the expression of heterologous genes.

Advantageously, Myrothecium host cells were found easy to transform, easy to culture, had a high growth rate coupled to high biomass production and were found to be suitable for growth in fermentors such as for large scale or industrial production of proteins of interest, including but not limited to enzymes such as amylases and xylanases or therapeutic drugs. Applications include but are not limited to protein and/or enzyme production for food and/or therapeutic applications. Another application concerns the use of a genetically engineered (transformed) Myrothecium strain as source of biopesticide.

In an embodiment of the invention, the DNA construct or nucleic acid construct integrates into the host cell chromosome. Alternatively, it may be present on an episome such as a plasmid.

The DNA construct or nucleic acid construct is built to allow modified expression (such as overexpression) of a homologous gene and/or may be built to allow expression of a heterologous gene. The protein produced by the transformed Myrothecium strain may be a Myrothecium protein, a fungal protein as well as a protein normally produced by another organism (i.e. non-fungal). The gene coding for the protein of interest may be engineered and or its codon-use adapted to further increase protein expression or may be engineered to incorporate mutations resulting in altered proteins.

The DNA construct or nucleic acid construct, if needed, comprises at least one operably linked tool that allows or enhances protein expression, said tool being selected from the group consisting of a promoter, a terminator, a polyadenylation signal, a leader, a secretion signal, a selection marker or reporter gene. Said tool may be of heterologous or homologous origin. It falls within the skills of an artisan to define type, multitude and sense of the genes and/or tools in the expression construct that give rise to optimal protein expression.

Preferred selection marker genes are the hygromycine B resistance gene, the phleomycin resistance gene, the phosphinothricine resistance gene, the acetamidase gene, a pyrG gene, an argB gene, a niaD gene and a trpC gene.

A preferred reporter gene is the gene coding for the TAKA-amylase of Aspergillus oryzae, optionally associated with flanking regions of the coding region from said TAKA-amylase gene or with flanking regions of the coding region from others genes.

Preferred promoters are the Aspergillus oryzae TAKA-amylase promoter, the Rhizomucor miehei aspartic proteinase promoter, the A. niger gluocamylase promoter, the A. niger neutral α-amylase promoter, the A. niger acid stable α-amylase promoter, the R. miehei lipase promoter and the promoters of the glycolytic enzymes genes GPD, PGK and ADH.

Said promoter may be a regulatable/inducible promoter or a constitutive promoter.

Suitable Myrothecium sp. include Myrothecium inundatum, Myrothecium prestonii, Myrothecium leucotrichum, Myrothecium cinctum, Myrothecium masonii, Myrothecium roridum, Myrothecium verrucaria, Myrothecium carmichaelii, Myrothecium lachastrae, Myrothecium atrum, Myrothecium atroviride, Myrothecium gramineum (syn. Xepiculopsis graminea).

Especially the Myrothecium gramineum strain MUCL39210, the Myrothecium gramineum strain CBS449.71, the Myrothecium gramineum IMI140595, the Myrothecium gramineum IMI290405 and the Myrothecium verrucaria strain CBS328.52 were found highly suitable hosts for increased protein production, more in particular large-scale or industrial protein production.

An embodiment of the invention relates to Myrothecium cells transformed to comprise a PCNS43 or a p3SR2 vector.

The invention further relates to the use of the above-described Myrothecium transformants. They are very suitable for the use as cell factory, in particular for the production of proteins and/or enzymes. Possible applications include industrial protein and/or enzyme production, and therapeutical drugs production. Another aspect of the inventions concerns the use of Myrothecium transformants as source of biopesticide.

Another aspect of the present invention concerns methods of transforming Myrothecium sp., said methods comprising the steps of growing Myrothecium cells or protoplasts; introducing into said cells or protoplasts at least one recombinant DNA construct for the modulated expression of homologous genes and/or for the expression of heterologous genes into Myrothecium cells; and then selecting transformed i.e. genetically modified Myrothecium cells. Suitable transformation techniques include PEG-mediated transformation, electroporation, particle bombardment and/or Agrobacterium-mediated transformation. These and other suitable transformation techniques are well known in the art. The DNA construct with which to transform the Myrothecium host cells may be a plasmid or a vector.

Another aspect of the invention relates to isolated transformed or genetically modified Myrothecium strains obtainable by a method according to claim 18. These transformants or genetically modified strains preferably are characterized by an increased amylase activity, increased xylanase activity, increased growth rate, increased biomass production and/or reduced protease production. Advantageously, the transformants or genetically modified strains will have, after transformation with a suitable DNA construct or nucleic acid construct, an altered metabolic pathway compared to the non-transformed Myrothecium strain.

A further aspect of the present invention concerns a method for producing a protein of interest, said method comprising the steps of culturing transformed or genetically modified Myrothecium host cells of the invention under conditions which permit expression of the protein and then recovering the proteins of interest from said Myrothecium culture. Said protein preferably is a fungal protein such as a fungal enzyme but may as well be any other protein that can be expressed in Myrothecium sp.

A still further aspect of the present invention concerns a method for producing the biomass of the transformed or genetically modified Myrothecium host cells, said method comprising the steps of culturing said host cells and recovering them with or without the culture medium. Said method thus optionally comprises a purification step to separate Myrothecium cells from culture medium or other compounds.

### SHORT DESCRIPTION OF THE DRAWINGS

FIG. 1 shows a phylogenetic tree constructed with fungal ITS sequences available in the NCBI database or provided in the present invention.

FIG. 2 shows a partial nucleotide sequence and its deduced protein sequence of the Myrothecium gramineum MUCL39210 glyceraldehyde-3-P dehydrogenase gene

FIG. 3 shows a multiple sequence alignment of four partial GPD protein sequence from the strains MUCL39210, MUCL11831, CBS449.71 and IMI290405.

FIG. 4 shows an agarose gel electrophoresis of the DNA fragments amplified by PCR from Myrothecium transformants and corresponding to a part of the Aspergillus oryzae α-amylase reporter gene (odd lanes from 5 to 13) and to a part of the hygromycin selection gene (even lanes from 4 to 12). Lanes 2-3 and 14-15 correspond respectively to negative and positive controls.

FIGS. 5A-E show the 28s rDNA sequence of the strains MUCL39210 (A), MUCL11831 (B), CBS449.71 (C), IMI140595 (D) and IMI290405 (E).

FIGS. 6A-E show the ITS sequences of the strains MUCL39210 (A), MUCL11831 (B), CBS449.71 (C), IMI140595 (D) and IMI290405 (E).

FIGS. 7A-C show the DNA sequences of the gpd gene of the strains MUCL11831 (A), CBS449.71 (B) and IMI290405 (C).

FIG. 8 shows a consensus sequence of the gpd gene of the strain MUCL39210.

FIG. 9 shows the amino acid sequence of the glyceraldehyde 3-P dehydrogenase of Myrothecium gramineum (Xepiculopis graminea) MUCL39210.

### DETAILED DESCRIPTION OF THE INVENTION

A first aspect of the invention is the screening of a collection of fungi. The collection screened in the present invention is the collection from the MUCL (“Mycothèque de l'Université Catholique de Louvain”, Place Croix du Sud, 2, B-1348 Louvain-la-Neuve, Belgium). However, it will be obvious for a person skilled in the art that other public or private microorganisms collections can be screened following the same approach to provide the selected genus with the required properties. Examples of public collections where fungal strains are available are ATCC (American Type Culture Collection—P.O. Box 1549, Manassas, Va. 20108, USA), CBS (Centraalbureau voor Schimmelcultures—P.O. Box 85167, 3508 AD Utrecht, The Netherlands), DSMZ (Deutsche Sammlung von Mikroorganismen und Zellkulturen GmbH—Mascheroder Weg 1 b, D-38124 Braunschweig, Germany), IMI (International Mycological Institute—Bakeham Lane, Egham, Surrey TW20 9TY, UK), . . . .

3.000 different species or strains of fungi were first selected from the MUCL collection based on several criteria such as but not restricted to their non-pathogenicity or the fact that no transformation for this species has been reported in the scientific literature or in a patent. All these strains were grown in liquid culture and on solid medium. Useful liquid media are rich media containing for example glucose, peptone, yeast extract, malt extract, soy meal, . . . . Useful solid media are for example PDA (Potato Dextrose Agar), MA (Malt Extract Agar). The first selection criteria were based on the growth rate, the biomass production, the protein production, the enzyme production (xylanase, amylase and proteases) and the morphology of the fungal strains.

The growth rate can be usefully determined for example by measuring the rate of increase of the growth diameter of a single colony on a solid medium.

The biomass production can be usefully determined by evaluating for example the dry matter of cells collected by filtration or by centrifugation at various time intervals during the culture.

The protein production can be usefully determined, using methods well known by persons skilled in the art, by measuring the total amount of proteins in a culture supernatant. Examples of such methods are the Lowry (Lowry O. H. et al. 1951. J. Biol. Chem. 193:265) or the Bradford (Bradford M. M. 1976. Anal. Biochem. 72:248) methods.

The enzyme production can be usefully determined by measuring the amount of enzyme produced in a culture supernatant using methods well known by persons skilled in the art. Examples of methods of xylanase activity determination are the measure of the release of reducing sugar from xylan (Miller G. L. 1959. Anal. Chem. 31:426) or the measure of the release of coloured compounds from modified substrates (for examples AzoWAX or Xylazyme AX from Megazyme). Amylase activity can be determined for example by measuring the rate of release of reducing sugar from starch (Miller, op. cit.) or by using specific commercially available kits (amylase kit from Sigma; Amyl kit from Roche Diagnostics, . . . ). Protease activity can be determined for example using commercially available kits (Azocasein from Megazyme, . . . ).

The morphology of the fungal strains can be advantageously studied by examining portions of the cultures with a microscope (light microscope, electronic microscope, . . . ).

From this screening were retained about one tenth of the 3.000 fungal species or strains. Among the selected strains, about 80 strains were chosen based on their good protein production, about 80 based on their good biomass production, about 60 based on their good amylase production, about 60 based on their good xylanase production and about 10 based on their good or bad protease production.

In one aspect it may be useful to obtain strains that produce very low levels of proteases. This reduces the risk of proteolytic degradation of proteins of interest possibly produced from introduced heterologous genes. Alternatively, good protease producers might be of interest with the purpose of isolating and using for instance a promoter thereof to enhance protein and/or enzyme production in other strains. In one aspect it may be useful to obtain strains that produce very low levels of proteases. This reduces the risk of proteolytic degradation of proteins of interest possibly produced from introduced heterologous genes. Alternatively, good protease producers might be of interest with the purpose of isolating and using for instance a promoter thereof to enhance protein and/or enzyme production in other strains.

The selected strains were submitted to a second round of screening in liquid cultures using five different culture media, each one having a particular purpose. The first medium has been selected for maximal protein production, the second for maximal biomass production, the third for the amylase induction, the fourth for xylanase induction and the last one for the protease induction.

Typical media for protein and/or biomass production are rich media composed for example of carbon and nitrogen sources such as glucose, malt extract, peptone, soy meal, . . . . A typical medium for amylase production contains usually starch or starch derivatives as enzyme inducer. A typical medium for xylanase production usually contains an hemicellulose-rich inducer such as but not restricted to xylan, wheat straw or wheat bran. A typical medium for protease production is usually enriched in various proteins or amino acids sources.

The screening criteria retained were the same as for the first step: the growth rate, the biomass production, the protein production, the production of amylase, xylanase and/or protease and the morphology.

A statistical approach was followed to identify the strains that ranked with a high score for one or several criteria in more that one culture medium. About 50 strains were selected out from this second screening.

In order to eliminate those strains that would produce any antimicrobial compounds, the production of such compounds by the selected strains has been checked. The inhibition of growth of some bacterial species has been tested using the method described by Färber et al (Färber P. and Geisen R. 1994. Antagonistic activity of the food-related filamentous fungus Penicillium nalgiovense by the production of penicillin, Appl. Environ. Microbiol. 60:3401-3404). Indicator strains were the following: Micrococcus flavius (MUCL collection, determined with API 20E), Bacillus subtilis DSM6633, Bacillus cereus LMG6923, Staphylococcus aureus, Salmonella sp. (MUCL collection, determined with API 20E), Escherichia coli, Proteus mirabilis (MUCL collection, determined with API 20E), Candida blankii MUCL29808. These strains have been chosen according to Färber et al (op. cit.), to Aissaoui & al (Aissaoui H., Agut M., Torras M. 1999. Effect of the raw extract of Arthrinium strains (Hyphomycetes, Dematiaceae) on the growth of pathogenic bacteria in poultry feed. Microbios. 100(396):109-115) and to the Oxoid Manual (7^{th} edition 1995). It is also obvious for a person skilled in the art that other bacterial species or strains can equally be used for the evaluation.

Potential producers of antibacterial compounds have been identified and discarded from the list of selected strains.

A similar method has been used to control the production of antifungal compounds using Botrytis cinerea MUCL30834, Fusarium moniliforme MULC14280 and Alternaria alternata MUCL16089 (Mangiarotti A., Frate G., Picco A M., Caretta G. 1987. Antagonistic activity in vitro of some saprophytic fungi occurring on the phylloplane of rice, wheat and maize. Boletin Micologico. 3:183-189). Again the potential antifungal compounds producers were discarded from the list.

30 of the remaining strains (the best ranking in the statistical analysis) were then tested for their ability to incorporate foreign DNA as well as to express a reporter gene. The general method of protoplasts transformation (Punt P. J. and van den Hondel C. A. M. J. J. 1992. Transformation of filamentous fungi based on hygromycin B and phleomycin resistance markers, Methods in Enzymology. 216:447-457) has been followed. For each strain several parameters, that a person skilled in the art will recognise as critical, were investigated. Among these parameters were:
the possibility to use one or several of the common transformation markers (hygromycine B resistance gene, phleomycin resistance gene, phosphinothricine resistance gene, acetamidase gene, pyrG gene, argB gene, niaD gene, trpC . . . )
the conditions to obtain viable protoplasts in large amounts (culture medium, culture temperature, length of the culture, type of digesting enzymes, duration of cell walls digestion, type of osmotic stabiliser, . . .
the conditions of transformation.
the conditions of protoplasts regeneration.

A useful reporter gene is the well-known gene coding for the TAKA-amylase of Aspergillus oryzae. The flanking regions of the coding region (essentially the promoter and the terminator sequences) or flanking regions of the coding region from others genes are advantageously associated with the coding region to provide a complete expression cassette. The sequence of the TAKA-amylase gene is publicly available (Tada, S., Iimura, Y., Gomi, K., Takahashi, K., Hara, S. and Yoshizawa, K. 1989. Cloning and nucleotide sequence of the genomic Taka-amylase A gene of Aspergillus oryzae. Agric. Biol. Chem. 53:593-599) and the corresponding DNA can be easily cloned using for example PCR techniques.

It is therefore the object of the present invention to show that the genus Myrothecium presents all the characteristics needed for a performing cloning host such as but not limited to good transformability, good expression of homologous and heterologous genes or genes sets, easy metabolic pathway engineering, good performances in fermentation, . . . .

In a specific aspect of this embodiment, it is more precisely shown that the species Myrothecium gramineum (syn. Xepiculopsis graminea) presents all the characteristics needed for a performing cloning host such as but not limited to good transformability, good expression of homologous and heterologous genes or genes sets, easy metabolic pathway engineering, good performances in fermentation, . . . .

In a more specific aspect of this embodiment, it is more precisely shown that the strain Myrothecium gramineum (syn. Xepiculopsis graminea) MUCL39210 presents all the characteristics needed for a performing cloning host such as but not limited to good transformability, good expression of homologous and heterologous genes or genes sets, easy metabolic pathway engineering, good performances in fermentation, . . . .

The genus Myrothecium is defined in the following way:

“Fructification cupulate, sporodochial or synnematous, formed from closely compacted conidiophores arising from a more or less developed stroma and bearing a mass of slimy green to black spores which becomes hard on drying. Fructification surrounded by differentiated marginal hyphae which may be free or laterally compacted into a plectenchymatous wall. Hyaline or darkened setae sometimes present arising from the basal stroma. Coniodiophores hyaline, olivaceous or slightly darkened, macronematous, irregularly and repeatedly branched forming several branches at each node, the ultimate branches bearing the conidiogenous cells (phialides) in whorls. Phialides hyaline or darkened at the apex, occasionally percurrent sometimes with a flared collarette, compacted into a dense parallel layer. Spores unicellular, hyaline or dilute olivaceous, black in mass, slimy” (Tulloch M., 1972, The genus Myrothecium Tode ex Fr. Mycological Papers no 130, Commonwealth Mycological Institute, Kew, Surrey, England).

The genus Myrothecium includes i.a. the species Myrothecium inundatum, Myrothecium prestonii, Myrothecium leucotrichum, Myrothecium cinctum, Myrothecium masonii, Myrothecium roridum, Myrothecium verrucaria, Myrothecium carmichaelii, Myrothecium lachastrae, Myrothecium atrum, Myrothecium atroviride, Myrothecium gramineum, . . . .

The species Myrothecium gramineum or its synonymous species Xepiculopsis graminea has the following description:

“Foliicolous. Conidiomata stromatic, scattered to gregarious, seemingly superficial, discoid to cupulate, excipulate with a dense white fringe of long white setae and sterile elements with coiled apices surrounding a central greenish-black agglutinated conidial mass, 220-450 μm diam., 150-190 μm deep (excluding setae); basal stroma moderately developed, composed of a colourless textura angularis in the lower layers gradually merging with textura prismatica above; excipulum well-developed, of compact and colourless textura prismatica to textura porrecta; marginal hyphae with terminal cells 7-16×2-3 μm, of two kinds: those on the inside subcylindrical with blunt apices, olivaceous to fuliginous, irregularly tuberculate, and those on the outside with a subcylindrical, olivaceous to fuliginous, irregularly tuberculate basal part, and a slender, coulourless, smooth, sinuate apical process up to 47 μm long and 1 μm wide.

Conidiomatal setae sparse arising from outer cells of the excipulum, unbranched, straight or slightly incurved, subulate, thick-walled with continuous narrow lumen, smooth-walled, colourless, up to 450 μm long, 15-20 μm wide at the broadest point, up to 1.5 μm wide at the acute apex. Conidiophores arising from the uppermost cells of the basal stroma and inner layer of cells of the excipulum in the concavity of the conidiomata, unbranched or branched, septate, colourless to almost colourless, smooth. Conidiogenous cells discrete, occasionally integrated, subcylindrical to clavate with flared collarettes and moderate to marked apical periclinal thickenings, more or less pale olivaceous in the upper part with a deeper pigmentation near the collarettes, smooth, (7-)12-16×2-3 [mean=14×2.5] μm. Conidia fusiform to ellipsoid with an acute apex and a narrow truncate base, unicellular, pale olivaceous, smooth, 6.5-12.5×2-3(−3.5) [mean=9×2.5] μm, with an apical mucoid appendage; mean conidium length/width ratio=3.6:1. (NAG RAJ T. R. 1993. Coelomycetous anamorphs with appendage-bearing conidia, published by Mycologue Publication)

It should be understood that in the present invention the terms Myrothecium sp., Myrothecium gramineum or Xepiculopsis graminea refer not only to those organisms present currently under these names in the various public or private microorganisms collections around the world but also to the species which have previously been or currently are assigned to other species but that possess the same morphological and cultural characteristics as defined above.

The scope of the present invention also includes mutant strains of Myrothecium sp., Myrothecium gramineum or Xepiculopsis graminea. Types of mutants include but are not restricted to auxotrophic mutants, protease(s) deficient mutants, secretion mutants, constitutive mutants, deregulated mutants. Mutants can be obtained using techniques well known by persons skilled in the art.

In another embodiment of this invention, it will be referred to rDNA sequences or ITS sequences to assign a particular strain to a species or a genus.

The internal transcribed spacer (ITS) region of nuclear ribosomal DNA (nrDNA) has been widely used to reconstruct microbial phylogeny. The ITS region separating 18s and 26s nrDNA and the 5.8s coding region sequence has been widely used to characterise interspecific and intergenic levels divergences. The ITS region is therefore useful to gain insight into DNA sequence evolution. This is due to the fact that, as a transcribed, but untranslated sequence, it is free to vary.

Using a combination of the morphological taxonomical methods and the ITS sequences determination and comparison, the inventors have shown that some of the strains deposited as Myrothecium gramineum (or as its synonymous Xepiculopsis graminea) in public culture collections have been erroneously identified as Myrothecium gramineum or Xepiculopsis graminea. They have also shown that representative members of other species of the genus Myrothecium also present the characteristics needed for a performing cloning host such as but not limited to good transformability, good expression of homologous and heterologous genes or genes sets, easy metabolic pathway engineering, good performances in fermentation, . . . .

Other species of the genus Myrothecium are therefore considered as integral part of the present invention.

Aspergillus species and in particular Aspergillus niger have been used since years as cell factories to produce homologous and heterologous proteins and can therefore be considered as the models for gene expression in filamentous fungi (see for example the review van Gorcom R. F. M., Punt P. J., van den Hondel C. A. M. J. J. 1994. Heterologous gene expression in Aspergillus in The genus Aspergillus: from taxonomy and genetics to industrial applications Powell K. A., Renwick, A., Peberdy J. F. eds. Plenum Press, New York, pp 241-250).

It is therefore one of the objects of the present invention to show that strains from the selected filamentous fungi genus produce, when transformed with homologous or heterologous DNA more protein and/or enzyme than Aspergillus niger in the same conditions.

It is a further object of the present invention to provide nucleic acid or DNA constructs (recombinant DNA constructs) that, when used to transform Myrothecium sp., Myrothecium gramineum or Xepiculopsis graminea, allow the expression of the homologous or the heterologous gene present on this nucleic acid or DNA construct. The foreign (introduced) DNA may integrate into the host genome or may be present on an episome. A particular result of the present invention is that the DNA when introduced in the host cell, integrates in the host cell chromosome. This phenomenon is similar to what has already been described for many other species of fungi. Therefore, it is obvious for a person skilled in the art that the same type of constructs as the one used for other species of filamentous fungi can be used for Myrothecium sp., Myrothecium gramineum or Xepiculopsis graminea. For example, the DNA constructs that are useful in the transformation of Aspergillus species such as Aspergillus niger, Aspergillus nidulans or Aspergillus oryzae are also useful for the purpose of the present invention. The promoter may be any DNA sequence that induces a transcriptional activity in the host and may be derived from any homologous or heterologous gene. Examples of promoters are the Aspergillus oryzae TAKA-amylase promoter, the Rhizomucor miehei aspartic proteinase promoter, the A. niger gluocamylase promoter, the A. niger neutral α-amylase promoter, the A. niger acid stable α-amylase promoter, the R. miehei lipase promoter and the promoters of glycolytic enzymes genes such as GPD, PGK, ADH. These promoters may be regulatable/inducible or not. Transcription terminator and polyadenylation sequences may also be added to the nucleic acid or DNA constructs.

In a particular aspect of this embodiment, it is also provided additional tools to enhance the gene expression in the selected host strains, namely Myrothecium sp., Myrothecium gramineum, or Xepiculopsis graminea. Using methods well known by persons skilled in the art, the gene coding for the glyceraldehyde 3-phosphate dehydrogenase (GPD gene) has been partially cloned. Flanking regions (promoter and terminator) of these sequences highly suited to make nucleic acid or DNA constructs that give a better expression in the selected host strains.

In a further aspect of this embodiment, the nucleic acid or DNA constructs may comprise particular sequences, such as sequences coding for a leader or a signal peptide, that allows the protein of interest to be secreted in the extracellular medium. This preregion may be derived from any secreted protein, or may be synthesised using consensus sequences, provided that this preregion allows the secretion of the desired protein.

In yet another aspect of this embodiment, the nucleic acid or DNA constructs may comprise genes to allow the selection of the transformants of Myrothecium sp., Myrothecium gramineum or Xepiculopsis graminea. Those selection markers are for example but are not restricted to the acetamidase gene (amdS) of Aspergillus sp. or the hygromycine resistance gene (hygB) of Escherichia coli. Other markers include the argB, niaD, trpC or pyrG genes of Aspergillus sp.

In yet another aspect of this embodiment, the various elements of the DNA constructs may be either incorporated in the same vector or plasmid or DNA fragment or may be included in separate vectors, plasmids or DNA fragments provided that the DNA sequences required for gene expression are associated with their respective coding sequence.

An example of suitable selection vector is the plasmid pCSN43 that contains the hph gene of Escherichia coli under the control of Aspergillus transcriptional signals (Staben C. et al. 1989. Use of a bacterial hygromycine B resistance gene as a dominant selectable marker in Neurospora crassa transformation. Fungal Genetics Newsl. 36:79). It confers to transformed or genetically modified cells resistance to hygromycine B.

Another example of suitable selection vector is the plasmid p3SR2 that contains the acetamidase gene from Aspergillus nidulans (Hynes et al. 1983. Isolation of genomic clones containing the amdS gene of Aspergillus nidulans and their use in the analysis of structural and regulatory mutations. Mol. Cell. Biol. 3:1430). It confers to transformed or genetically modified cells the ability to grow on a minimal medium containing acetamide as sole nitrogen source.

In another embodiment of the present invention, a method is provided to transform Myrothecium sp., Myrothecium gramineum or Xepiculopsis graminea. The preferred method of transformation is the standard protoplast transformation. In this method, the DNA constructs are incubated in particular conditions with protoplasts of the host strain. The transformants are first selected on the basis of the selection marker used (for example amdS or hygB) and in a second step cotransformants that have integrated the desired gene are screened.

It is one of the objects of the present invention to show that, when transformed or genetically modified with two separate DNA constructs (containing for example a selection marker and a reporter gene), most of the transformants of Myrothecium, Myrothecium gramineum or Xepiculopsis graminea have integrated simultaneously both DNA constructs.

Other methods of transformation can usefully replace the protoplasts transformation and are well known by persons skilled in the art. Examples of alternative methods are the electroporation, the transformation by particle bombardment or the transformation by Agrobacterium sp.-mediated recombination.

In another embodiment of the present invention, it is provided Myrothecium sp., Myrothecium gramineum or Xepiculopsis graminea transformed or genetically modified strains that express or overexpress a particular homologous or heterologous gene or a particular set of genes.

In another embodiment of the present invention, it is provided Myrothecium sp. Myrothecium gramineum or Xepiculopsis graminea genetically engineered strains with one or several altered metabolic pathways.

In another embodiment of the present invention it is provided culture media to cultivate the Myrothecium sp., Myrothecium gramineum or Xepiculopsis graminea transformed or genetically modified strains. A typical culture media contain carbon (C) sources and/or nitrogen (N) sources and/or salts and/or secondary metabolites and/or miscellaneous compounds. C sources could be glucose, saccharose, . . . as well as complex substrates like arabinoxylan, pectin, . . . as well as lipids and organic acids. N sources could be amino acids, nitrate, ammonia, . . . as well as complex substrates like yeast extract, potato extract, soy extract, . . . .

Salts could be chosen among phosphate salts, Magnesium salts, potassium salts, . . . . Secondary metabolites could be antibiotics, mycotoxins, hormones, vitamins, . . . . Miscellaneous compound could be defoamers, antifoamers, emulsifiers, . . . .

In another embodiment of the present invention it is provided culture techniques to cultivate the Myrothecium sp., Myrothecium gramineum or Xepiculopis graminea or genetically modified strains. Examples of such culture techniques are liquid cultures in erlenmeyer flasks or fermentor, solid state fermentation, airlift fermentation, . . . .

This invention will be described in further details in the following examples by reference to the enclosed drawings, which are not in any way intended to limit the scope of the invention as claimed.

### EXAMPLE 1

Characterisation of the Myrothecium Strains

1.1. Molecular Characterisation of the Myrothecium Strains

The genomic DNA of the strains currently assigned as Myrothecium gramineum (or of the synonymous strain Xepiculopsis graminea) MUCL39210, MUCL11831, CBS449.71, IMI140595 and IMI290405 has been prepared using the Dneasy Plant Mini Kit (Qiagen).

The DNA coding for RNA ITS (Internal Transcribed Spacer) and 28S regions were analysed by PCR. The oligonucleotides used for the 28S region were: LR0R (5′-ACCCGCTGAACTTAAGC-3′, SEQ ID No 1), LR6 (5′-CGCCAGTTCTGCTTACC-3′, SEQ ID No 2), LR3R (5′-GTCTTGAAACACGGACC-3′, SEQ ID No 3) and LR3 (5′-CCGTGTTTCAAGACGGG-3′, SEQ ID No 4). The oligonucleotides used for the ITS region were ITS1 (5′-TCCGTAGGTGAACCTGCGG-3′, SEQ ID No 5) and ITS4 (5′-TCCTCCGCTTATTGATATGC-3′, SEQ ID No 6).

PCR reactions were performed in 0.25 ml tubes, using a MJ Research PTC-200 Peltier Thermal Cycler. PCR reactions (50 μl) contained approximately 20 ng of fungal genomic DNA (in 10 μl), 10 pM ITS1 and ITS4 primer pairs (1 ml), 100 mM dNTPs (1 μl), 1× reaction buffer (10 mM Tris-HCl, pH 8.3, 1.5 mM MgCl.sub.2, 50 mM KCl, 0.1 ng/ml BSA) (5 μl), and 2.5 U Taq polymerase (Invitrogen) (0.5 μl). Thermal cycling conditions were as follows: for 28S: initial denaturation (94° C., 5 min), followed by 35 cycles of denaturation (94° C., 1 min), annealing (50° C., 1 min 30 s), primer extension (72° C., 4 min), and one final cycle of primer extension (72° C., 10 min); for ITS: initial denaturation (94° C., 3 min), followed by 30 cycles of denaturation (94° C., 1 min 30 s), annealing (55° C., 1 min 30 s), primer extension (72° C., 2 min), and one final cycle of primer extension (72° C., 10 min).

The reaction products (10 μl) were analysed by electrophoresis on a 0.8% agarose gel in Tris-acetate EDTA buffer (TAE buffer) to check the presence of a unique amplified DNA fragment. The PCR product was then purified using PCR purification kit (Qiagen) and quantified with a Biophotometer (Eppendorf).

DNA sequencing reactions were performed in 0.25 ml tubes, using a MJ Research PTC-200 Peltier Thermal Cycler. PCR reactions (20 μl) contained 25 to 100 fmol of purified PCR product (0.5 to 10 μl), 1.6 μM of −47 sequencing primer (2 μl), 8 μl of DTCS Quick Start Master Mix (100 μl of dNTPs, 200 μl of ddATP, 200 μl of ddGTP, 200 μl of ddCTP, 200 μl of ddUTP, 200 μl of 10× reaction buffer and 100 μl Taq polymerase). Thermal cycling conditions were as follows: 30 cycles of denaturation (96° C., 20 sec), annealing (50° C., 20 sec), primer extension (60° C., 4 min), followed by holding at 4° C. The reaction is stopped by adding 4 μl of Stop Solution (1.5M NaOAc+50 mM EDTA prepared fresh daily by mixing equal volumes of 3M NaOAc and 100 mM EDTA) and 1 μl of 20 mg/ml glycogen. The sequencing reactions were transferred to a 96 wells plate. 60 μl of 95% cold ethanol from −20° C. freezer were added, the plate was sealed with adhesive foil, mixed thoroughly, incubated at −20° C. during 10 minutes, centrifuged at 14.000 rpm at 4° C. during 30 minutes and the supernatant was carefully removed. The pellets were then rinsed two times with 200 μl of 70% cold ethanol. Finally, the samples in the plate were dried under vacuum until no traces of ethanol remained (5 to 15 minutes). The samples were resuspended in 40 μl of Sample Loading Solution, left at room temperature for ten minutes then mixed thoroughly and overlaid with one drop of light mineral oil (Sigma Cat# M 5904).

The sequencing was done in a capillary electrophoresis sequencer “CEQ™ 2000 XL DNA Analysis System” (Beckman Coulter). The sequences generated were rectified and aligned with the software Sequencher™ 4.1. The sequence of 28s rDNA of the strain MUCL39210 is shown in SEQ ID No 7.

The sequence of 28s rDNA of the strain MUCL11831 is shown in SEQ ID No 8.

The sequence of 28s rDNA of the strain CBS449.71 is shown in SEQ ID No 9.

The sequence of 28s rDNA of the strain IMI140595 is shown in SEQ ID No 10.

The sequence of 28s rDNA of the strain IMI290405 is shown in SEQ ID No 11.

The ITS sequence of the strain MUCL39210 is shown in SEQ ID No 12.

The ITS sequence of the strain MUCL11831 is shown in SEQ ID No 13.

The ITS sequence of the strain CBS449.71 is shown in SEQ ID No 14.

The ITS sequence of the strain IMI140595 is shown in SEQ ID No 15.

The ITS sequence of the strain IMI290405 is shown in SEQ ID No 16.

These sequences were compared and compared with other closely related sequences of the NCBI gene bank (http://www.ncbi.nlm.nih.gov/BLAST/). The sequences were first aligned using the software ClustalX 1.5b and then a bootstrap tree was constructed using the software PAUP 4.0b10.

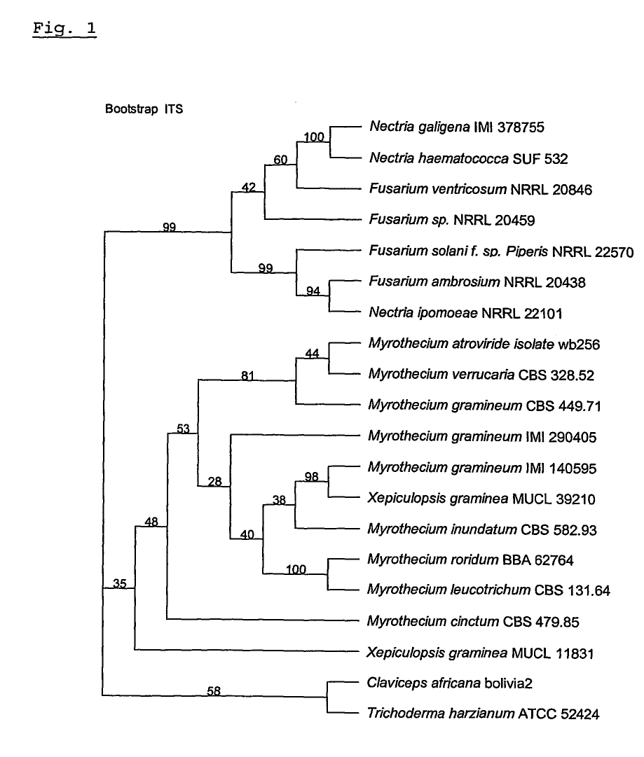

A phylogenetic tree with relevant data is presented on FIG. 1. It is concluded from this tree that some of the investigated Myrothecium gramineum (syn. Xepiculopsis graminea) strains have been erroneously assigned to a particular species. However, all the strains studied, with the exception of MUCL11831 belong to the Myrothecium genus.

1.2. Morphological Identification of the Myrothecium Strains.

A taxonomical reidentification of the MUCL39210 strain has confirmed that it presents all the morphological characteristics of the species Myrothecium gramineum (syn. Xepiculopsis graminea) (see the description in the detailed description of the invention).

A similar examination of the strain MUCL11831 confirmed that this strain does not belong to the Myrothecium genus.

### EXAMPLE 2

Transformation of Myrothecium

2.1. Choice of Selectable Markers

Myrothecium gramineum (syn. Xepiculopsis graminea) MUCL39210 was incubated on PDA (Potato Dextrose Agar), AMMA (Aspergillus Minimal Medium Agar) or MA2 (Malt Extract Agar) plates with increasing concentration of hygromycin B, phleomycin or phosphinothricine. The threshold antibiotic concentration where no growth occurred was 500 μg/ml for hygromycin B, >100 μg/ml for phleomycin and >400 μg/ml for phosphinothricin.

Myrothecium gramineum (syn. Xepiculopsis graminea) MUCL39210 was incubated on minimal medium plates containing acetamide as sole nitrogen source. The strain did not grow on this medium.

Similar results were obtained with the strains of Myrothecicum gramineum CBS449.71, IMI140595, IMI290405 and Myrothecium verrucaria CBS328.52

2.2. Vectors

2.2.a. Selectable Vector

The vector chosen to allow the selection of the transformants by their resistance to an antibiotic is the plasmid pCSN43 that contains the hph gene of Escherichia coli under the control of Aspergillus transcriptional signals (Staben C. et al. 1989. Use of a bacterial hygromycine B resistance gene as a dominant selectable marker in Neurospora crassa transformation. Fungal Genetics Newsl. 36:79).

The vector chosen to allow the selection of the transformed or genetically modified cells by their ability to grow on a minimal medium containing acetamide as sole nitrogen source is the plasmid p3SR2 (Hynes et al. 1983. Isolation of genomic clones containing the amdS gene of Aspergillus nidulans and their use in the analysis of structural and regulatory mutations. Mol. Cell. Biol., 3:1430)

2.2.b. Expression Vectors

2.2.b.1. p2G-S

A DNA fragment covering the coding region as well as its terminator region was amplified by a PCR reaction. The synthetic oligonucleotide AMY1 (5′-GGAATTCCACAGAAGGCATTTATG-3′, SEQ ID No 17) was chosen to contain the ATG codon of the first amylase methionine. The synthetic oligonucleotide AMY2 (5′-GCTCTAGAGCAACCACCAGGTCA-3′, SEQ ID No 18) corresponds to the sequence located about 400 bp downstream of the amylase terminal codon.

The two oligonucleotides have sequences recognised respectively by the restriction enzymes EcoRI and XbaI. Both primers (40 pmoles) were used for a PCR reaction with the gDNA from Aspergillus oryzae MUCL14492. The PCR reaction contained 2.5 U Pfu DNA polymerase (Stratagene), 20 mM Tris-HCl pH 8.0, 10 mM KCl, 2 mM MgCl₂, 6 mM (NH₄)₂SO₄, 0.1% Triton X-100, 1 μg BSA into a final volume of 100 μl. After denaturation of the DNA during 4 min at 94° C., the reaction was performed with 20 cycles of 30 s at 94° C., 30 s at 55° C. and 60 s at 72° C. followed by an elongation step of 7 min at 72° C. The amplified DNA fragments were purified in a final volume of 50 μl with the QIAquick PCR purification kit (Qiagen) according to the manufacturer's protocol. The purified fragment was treated with the EcoRI and XbaI restriction enzymes. The reaction was performed with 5 U Xba 1 and 5 U EcoR I (Pharmacia), 1× One-Phor-All buffer PLUS in 60 μl at 37° C. overnight. The restricted fragments were purified with the QIAquick gel extraction kit (Qiagen) after electrophoresis on an agarose gel and eluted into 30 μl water.

The PCR fragment was inserted between the EcoR I and Xba I sites of the pBluescript II SK(−) vector (Stratagene). The vector was prepared as follows: 0.5 μg pBluescript II SK was treated by 5 U EcoR I and 5 U Xba I restriction enzymes (Pharmacia) in a 20 μl final volume, 2× One-Phor-All buffer PLUS at 37° C. overnight. After electrophoresis in an agarose gel, it was purified with QIAquick gel extraction kit (Qiagen) and eluted in 30 μl water. 2 μl of the PCR fragment was ligated to this vector (1 μl) in the presence of 1 mM ATP, 1U T4 DNA ligase (Pharmacia) and 1× One-Phor-All buffer PLUS in a 10 μl final volume at 16° C. overnight. The ligation product (1 μl) was electroporated into competent DH10b cells (BRL-Gibco) after dialysis against water. The new plasmid was termed pAMY3.

The promoter of the glyceraldehyde-3-P dehydrogenase gene from Aspergillus nidulans was inserted in front of the amylase gene. This promoter allows a rather strong constitutive transcription of genes located downstream of it (Punt, P. J., Dingemanse, M. A., Kuyvenhoven, A., Soede, R. D. M., Pouwels, P. H. and van den Hondel; C. A. M. J. J., 1990. Functional elements in the promoter region of the Aspergillus nidulans gpdA gene coding for the glyceraldehyde-3-P dehydrogenase. Gene. 93: 101-109; Punt, P. J., Zegers, N. D., Busscher, M., Pouwels, P. H. and van den Hondel; C. A. M. J. J., 1991. Intracellular and extracellular production of proteins in Aspergillus under the control of expression signals of the highly expressed Aspergillus nidulans gpdA gene. J. Biotechnol. 17: 19-34). To insert the amylase gene downstream of the GPD promoter, the plasmid pFGPDGLAT2 was digested with the restriction enzyme NcoI. After purification with the QIAquick gel extraction kit (Qiagen), the plasmid was blunted with T4 DNA polymerase and dNTP, purified, and restricted with XbaI restriction enzyme (5U) in the presence of 1× One-Phor-All buffer PLUS in a 20 μl final volume 37° C. overnight). The same protocol was applied on pAMY3 except that the NcoI restriction enzyme was replaced by EcoRI. The fragments of interest were isolated after agarose gel electrophoresis, purified with QIAquick gel extraction kit (Qiagen) and collected separately in 30 μl water. The purified promoter and vector DNA fragment (1 μl) was ligated with the amylase gene and its terminator from pAMY3 (1 μl) in the presence of 1 mM ATP, 1U T4 DNA ligase (Pharmacia) and 1× One-Phor-All buffer PLUS in a 10 μl final volume at 16° C. overnight. The ligation product (1 μl) was electroporated into competent DH10b cells (BRL-Gibco) after dialysis against water. The new plasmid was named pGPD-AMY.

The GPD promoter, the amylase gene and its terminator haves been isolated from pGPD-AMY and inserted again into a new pGPD-AMY in order to have two copies of the functional gene on one plasmid. The first step was to digest pGPD-AMY with 1U HindIII restriction enzyme and 1× One-Phor-All buffer PLUS in a 40 μl final volume at 37° C. overnight. After purification with QIAquick gel extraction kit (Qiagen), the linear DNA fragment was blunted in the presence of T4 DNA polymerase and dNTP. The plasmid was again purified with QIAquick gel extraction kit (Qiagen) and treated with NotI restriction enzyme and 1× One-Phor-All buffer PLUS in a 40 μl final volume at 37° C. overnight. The fragment containing the functional amylase gene was finally isolated on agarose gel after electrophoresis. The pGPD-AMY plasmid (1 μg) was digested by XbaI (1U) and 1× One-Phor-All buffer PLUS in a 40 μl final volume at 37° C. overnight. After purification with QIAquick gel extraction kit (Qiagen), the linear DNA fragment was blunted in the presence of T4 DNA polymerase and dNTP. The plasmid was again purified with QIAquick gel extraction kit (Qiagen) and digested with the NotI restriction enzyme and 1× One-Phor-All buffer PLUS in a 40 μl final volume at 37° C. overnight. The vector with the DNA fragment containing a functional amylase gene was isolated after agarose gel electrophoresis. Both fragments were ligated together in the presence of 1 mM ATP, 1U T4 DNA ligase (Pharmacia) and 1× One-Phor-All buffer PLUS in a 10 μl final volume at 16° C. overnight. The ligation product (1 μl) was electroporated into competent DH10b cells (BRL-Gibco) after dialysis against water. The new plasmid was termed p2GPD-AMY.

Finally a selection marker was inserted in p2GPD-AMY. The acetamidase gene will allow the growth of fungi on a minimal medium with acetamide as unique nitrogen source. The functional acetamidase gene from A. nidulans (AmdS) has been isolated from 1 μg of p3SR2 plasmid digested with the restriction enzymesSalI (1U) and EcoRI (1U) and 2× One-Phor-All buffer PLUS in a 40 μl final volume at 37° C. overnight. This DNA fragment was inserted between the SalI and EcoRI restriction sites of pBluescript II SK. This new plasmid as well as p2GPD-AMY were digested with SalI (or Xba I for p2GPD-AMY), blunted by treatment with the T4 DNA polymerase and digested with NotI. The functional AmdS gene DNA fragment and the open p2GPD-AMY plasmid were isolated from agarose after gel electrophoresis. Both fragments were then ligated together in the presence of 1 mM ATP, 1U T4 DNA ligase (Pharmacia) and 1× One-Phor-All buffer PLUS in a 10 μl final volume at 16° C. overnight. The ligation product (1 μl) was electroporated into competent DH10b cells (BRL-Gibco) after dialysis against water. The new plasmid was termed p2G-S.

2.2.b.2. Cloning of the GPD Promoter

Design of the Oligonucleotides

Based on published sequences of fungal glyceraldehyde-3-P dehydrogenases (gpd), a set a four oligonucleotides has been designed in order to amplify a portion of the gpd gene of Myrothecium.

These oligonucleotides have the following sequences:

| | |
|---|---|
| Gpd1: | |
| 5′-GGNATCAAYGGITTCGG-3′, | SEQ ID No 19 |
| Gpd2: | |
| 5′-GTGSWGSWGGGGATGATGTT-3′, | SEQ ID NO 20 |
| Gpd3: | |
| 5′-GGTCGTATCGTNTTYCGIAAYGC-3′, | SEQ ID No 21 |
| Gpd4: | |
| 5′-GGAGCCAGGCAGTTGGTIGTRCA-3′, | SEQ ID No 22 |

Where N stands for A, T, C or G; Y stands for C or T; I stands for dInosine; S stands for G or C; W stands for A or T; R stands for A or G.
Preparation of the Genomic DNA

The genomic DNA of the following strains has been prepared using the Dneasy Plant Mini Kit (Qiagen): MUCL39210, MUCL11831, CBS449.71, IMI140595 and IMI290405.

PCR

PCR reactions have been performed in 0.25 ml tubes with a MJ research PTC-200 Peltier Thermal Cycler. The reaction mix (25 μl) contained about 20 ng genomic DNA, 10 pM oligonucleotides, dNTP 200 μM, 1× buffer (Tris-HCl 10 mM, pH 8.3, MgCl₂ 1.5 mM, KCl 50 mM, BSA 0.1 ng/ml) and 1 unit of Taq polymerase (Invitrogen). Amplification conditions were the following: initial denaturation (94° C., 4 min); 25 cycles of denaturation (94° C., 30 sec)-hybridisation (52° C., 30 sec)-elongation (72° C., 1 min); final elongation (72° C., 7 min).

Cloning of the gpd gene of the strains MUCL11831, CBS449.71 and IMI290405.

DNA fragments amplified using the protocol described above (with the oligonucleotides gpd1 and gpd2 as amplification primers) have been separated by agarose gel electrophoresis and purified with the aid of a QIAquick Gel Extraction Kit (Qiagen). A second round of PCR has been performed on these purified fragments and the resulting DNA fragments have been directly ligated into the plasmid commercially available pT-Adv (Advantage PCR cloning Kit—Clontech) using the recommendations of the manufacturer. Ligations products have been used to transform competent cells of Escherichia coli DH5α (Invitrogen). Positive clones have been identified by plasmid isolation and analysis by restriction enzyme digestion and agarose gel electrophoresis.

Sequencing of the gpd genes of the strains MUCL11831, CBS449.71 and IMI290405.

The insert of the plasmid described above has been sequenced using usual procedures.

The sequence of the gpd gene of the strain MUCL11831 is shown in SEQ ID No 23.

The sequence of the gpd gene of the strain CBS449.71 is shown in SEQ ID No 24.

The sequence of the gpd gene of the strain IMI290405 is shown in SEQ ID No 25.

Cloning of the gpd Gene of MUCL39210

The same procedure as for the cloning of the gpd genes of the strains MUCL11831, CBS449.71 and IMI290405, except that the first PCR was performed using the oligonucleotides gpd2 and gpd3 and that the second PCR was performed with the oligonucleotides gpd3 and gpd4.

Sequencing of the gpd Gene of the Strain MUCL39210.

The inserts of 8 different inserts of the plasmids obtained above have been sequenced using usual procedure.

A consensus sequence of the gpd gene of the strain MUCL39210 is shown in SEQ ID No 26.

Results

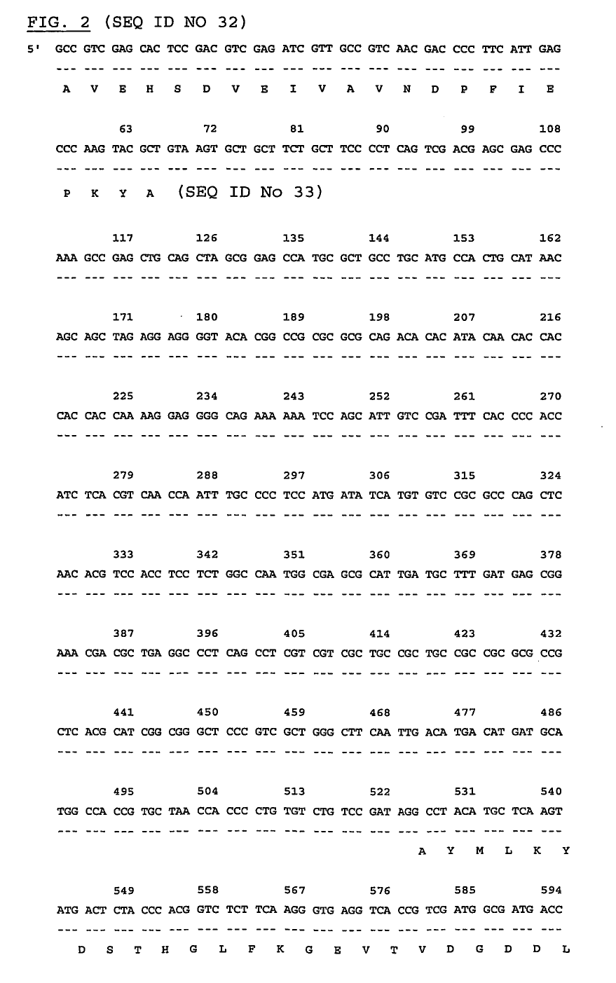

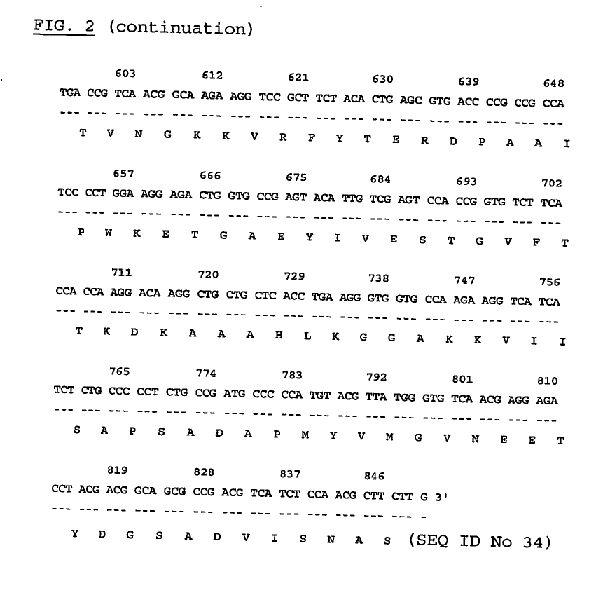

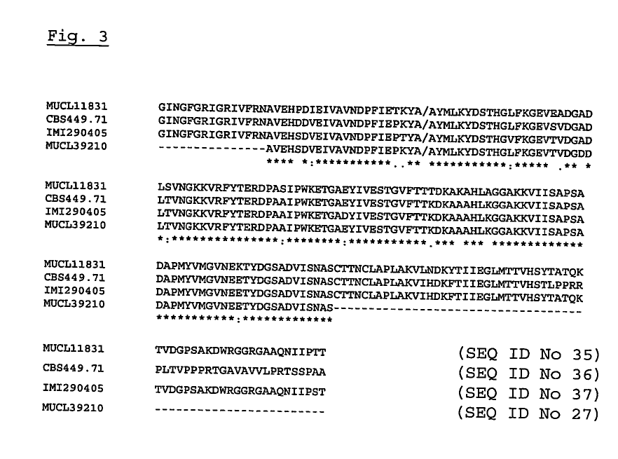

The MUCL39210 GPD partial sequence shown in SEQ ID NO14 was translated into a putative protein sequence as presented in FIG. 2. The GPD partial sequence comprises a large putative intron. The intron sequence does not show homology to any protein from protein databases when translated from the six possible DNA frames. The putative protein sequence (SEQ ID No 27) shows the highest similarity with the GPD protein sequence of Neurospora crassa using the BLASTP 2.2.4. program against the non-redundant GenBank database as of 20th Aug. 2002. Between the various Myrothecium GPD protein sequences, the identity is 88% as shown in FIG. 3.

2.3. Transformation

2.3.a. Transformation of Myrothecium

The strains Myrothecium gramineum (syn. Xepiculopsis graminea) MUCL39210, Myrothecium gramineum CBS449.71, IMI140595, IMI290405, and Myrothecium verrucaria CBS328.52 were transformed by generating protoplasts according to the protocol described by Punt et al. (Punt et al. 1992. Transformation of filamentous fungi based on hygromycin B and phleomycin resistance markers. Meth. Enzymol. 216:447).

The strains were grown in 350 ml Rich Liquid medium (malt extract 2%, peptone 1% and glucose 3%) at 25° C. for a period between 16 and 36 hours depending on the strain. The culture was filtered through a Miracloth filter to collect the mycelium. The mycelium was washed with the OSM solution (CaCl₂ 0.27 M, NaCl 0.6 M) and then incubated with 20 ml OSM solution/g mycelium supplemented with 12 mg/ml β-D-glucanase (Interspex), 7.5 mg/ml driselase (Interspex) and 81.4 U/ml yeast lytic enzyme (ICN). The incubation is done at 25° C. with slow agitation (80 rpm).

The apparition of the protoplasts is followed visually under the microscope and varies from 90 to 180 min, depending on the strain. Once the protoplasts were formed, the suspension was putted on ice. The protoplasts were separated from intact mycelium by filtration through a sterile Miracloth filter and diluted with 1 volume STC1700 solution (sorbitol 1.2 M, Tris-HCl pH 7.5 10 mM, CaCl₂ 50 mM, NaCl 35 mM) or GTC (glucose 1 M, CaCl₂ 50 mM, Tris 10 mM pH8). The protoplasts were then collected by centrifugation at 2000 rpm, 10 min, 4° C. and washed twice with STC1700 or GTC solution. They were finally resuspended in 100 μl of STC1700 or GTC (10⁸ protoplasts/ml).

To obtain transformants resistant to hygromycine, c.a. 2 μg pCSN43 plasmid DNA and c.a. 8 μg p2G-S plasmid DNA were added to the protoplasts suspension. To obtain transformants able to grow on acetamide as sole nitrogen source 3 μg p3SR2 plasmid DNA and 9 μg p2G-S plasmid DNA were added to the protoplasts suspension.

After 30 min at 20° C., 250, 250 and 850 μl PEG solution (PEG 4000 60%, Tris-HCl 10 mM pH 7.5 and CaCl₂ 50 mM) were added successively and the suspension was further incubated for 20 min at 20° C. PEG treated protoplasts suspension was diluted by the addition of 50 ml STC1700 or GTC and centrifuged 5 min at 4° C., 2000 rpm. The protoplasts were then resuspended in 200 μl STC 1700 or GTC and plated onto selective medium.

For hygromycin selection Malt Extract Agar Medium or Potato Dextrose Agar osmotically stabilised with 1.2 M sorbitol or 1 M glucose and supplemented with 500 μg/ml hygromycin was used. In some cases, protoplasts are plated onto the same medium without hygromycin. After 2 hours incubation, hygromycin (500 μg/l) overlays (8 ml molten agar) were dispensed over the protoplasts.

For acetamidase selection the AMM medium (Pontecorvo, G., Roper, J. A., Hemmons, L. M., Mc Donald, K. D. and Bufton, A. W. J., 1953. Adv. Genet. 5: 142-152.) supplemented with sorbitol 1.2M and acetamide as sole nitrogen source was used.

A series of transformants has been obtained for each strain and for each transformation trial. These transformants have been replicated on the same selection medium without sorbitol and kept for further analysis.

2.3.b. Control Transformation.

The same protocol as described in 2.3.a. has been used to transform the MUCL28817 strain of Aspergillus niger. A number of transformants have been selected and kept for further analysis.

### EXAMPLE 3

Analysis of the Transformants

3.1. Amplification of Integrated DNA

3.1.a. Preparation of Genomic DNA from Fungal Strains

The genomic DNA of the transformed or genetically modified fungal strains as well as the control strains has been prepared using the Dneasy Plant Mini Kit (Qiagen)

3.1.b. PCR Reactions

According to the published sequence of the E. coli hygromycin resistance gene (hph gene), the oligonucleotides hphpCSN431 (5′-ATGCCTGAACTCACCGCGACG-3′, SEQ ID No 28) and hphpCSN432 (5′-CTATTCCTTTGCCCTCGG-3′, SEQ ID No 29) were designed to amplify a region of 1020 bp comprising the hph gene.

To detect the presence of p2G-S into the Myrothecium genome without amplification of the endogenous amylase gene or the GPD promoter, the GPD2 (5′-TCTGGCATGCGGAGAG-3′, SEQ ID No 30) and AMY5 (5′-CGATGATGCCCTGCCA-3′, SEQ ID No 31) oligonucleotides were designed to allow the amplification of a 1134 bp length fragment covering a part of the A. niger GPD promoter fused to a part of the sequence of the TAKA amylase gene.

PCR reactions have been performed in 0.25 ml tubes with a MJ research PTC-200 Peltier Thermal Cycler. The reaction mix (25 μl) contained about 20 ng genomic DNA, 10 pM oligonucleotides, dNTP 200 μM, 1× buffer (Tris-HCl 10 mM, pH 8.3, MgCl₂ 1.5 mM, KCl 50 mM, BSA 0.1 ng/ml) and 1 unit of Taq polymerase (Invitrogen). Amplification conditions were the following: initial denaturation (94° C., 4 min); 25 cycles of denaturation (94° C., 30 sec)-hybridisation (55° C., 30 sec)-elongation (72° C., 1 min); final elongation (72° C., 7 min).

3.1.c. Results

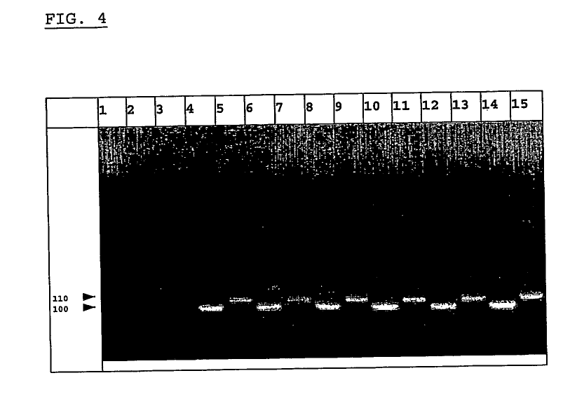

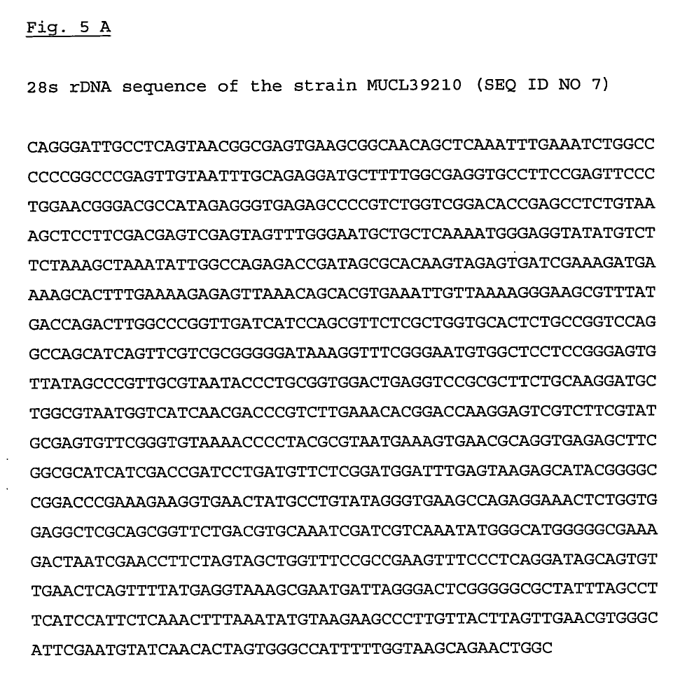

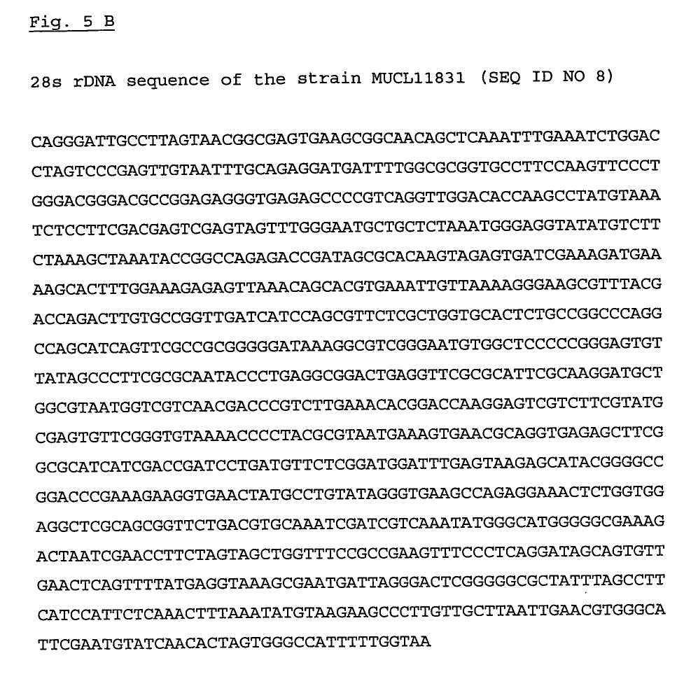

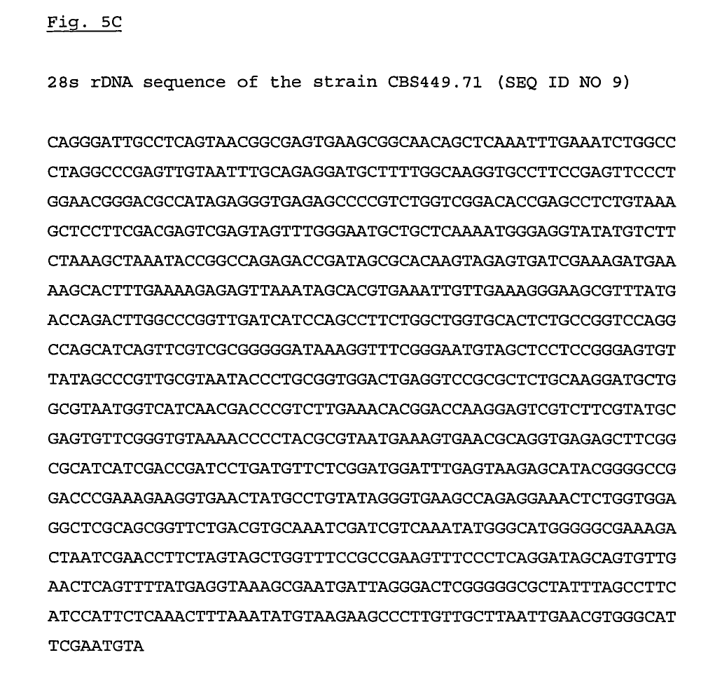

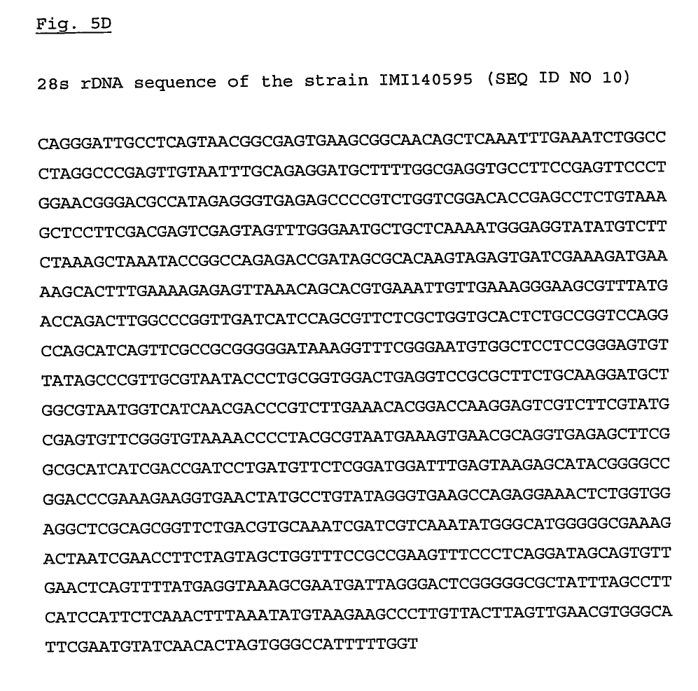

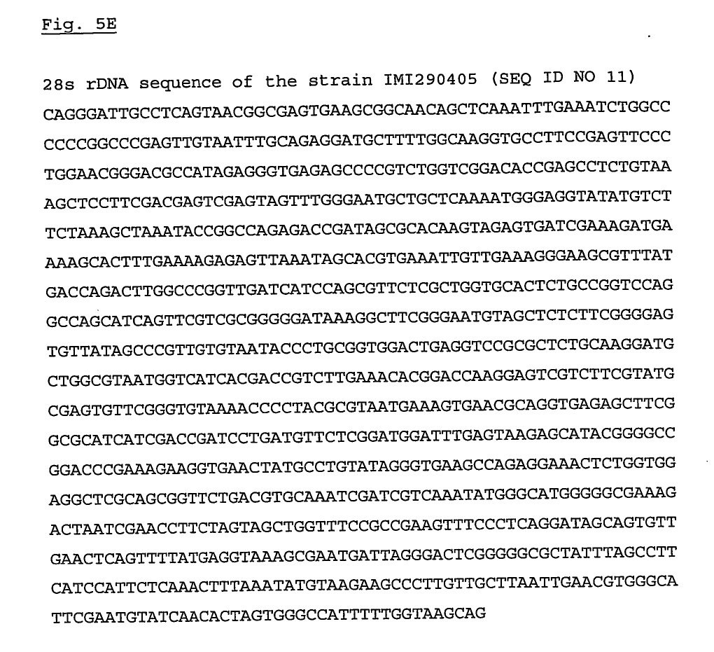

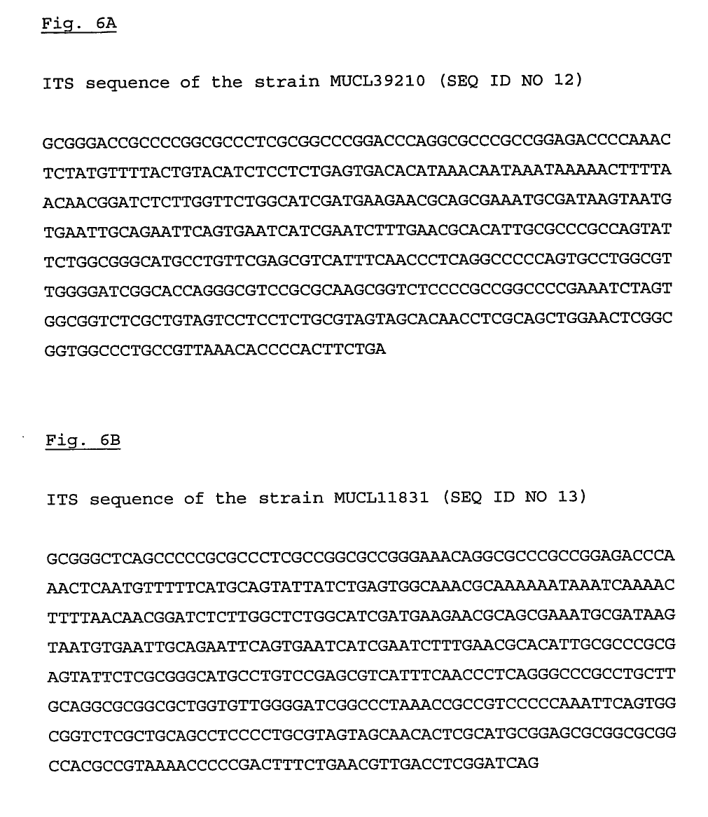

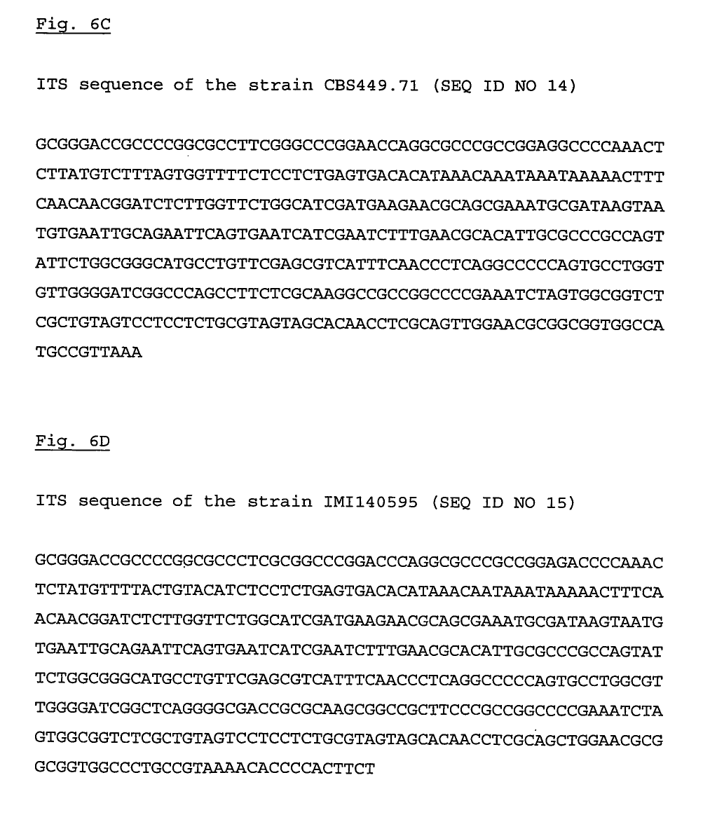

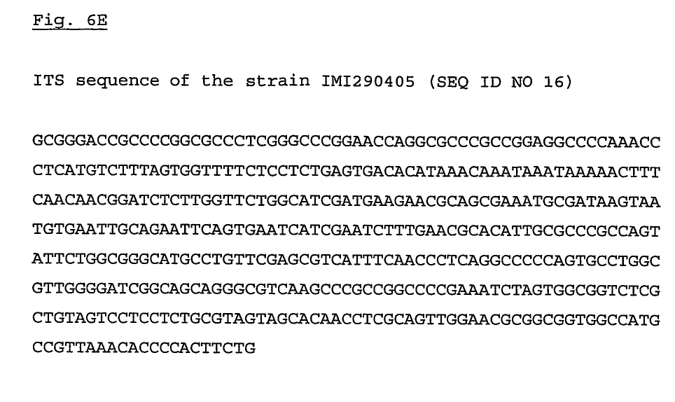

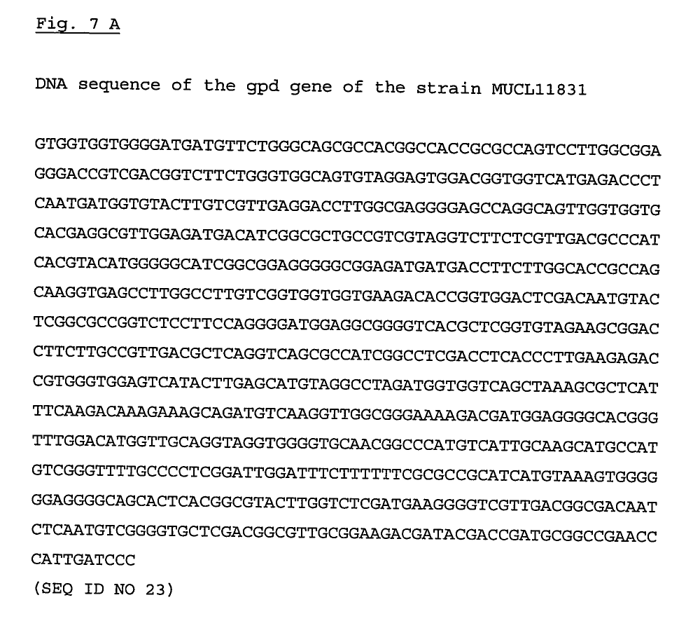

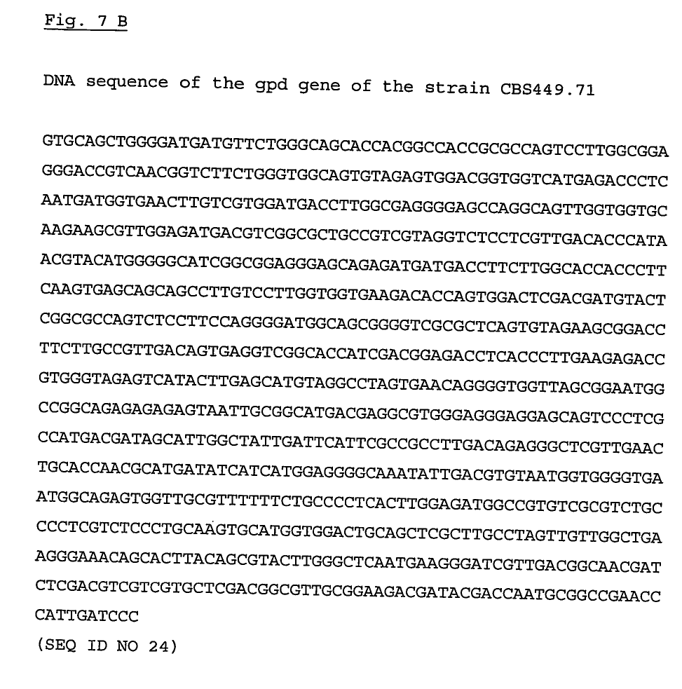

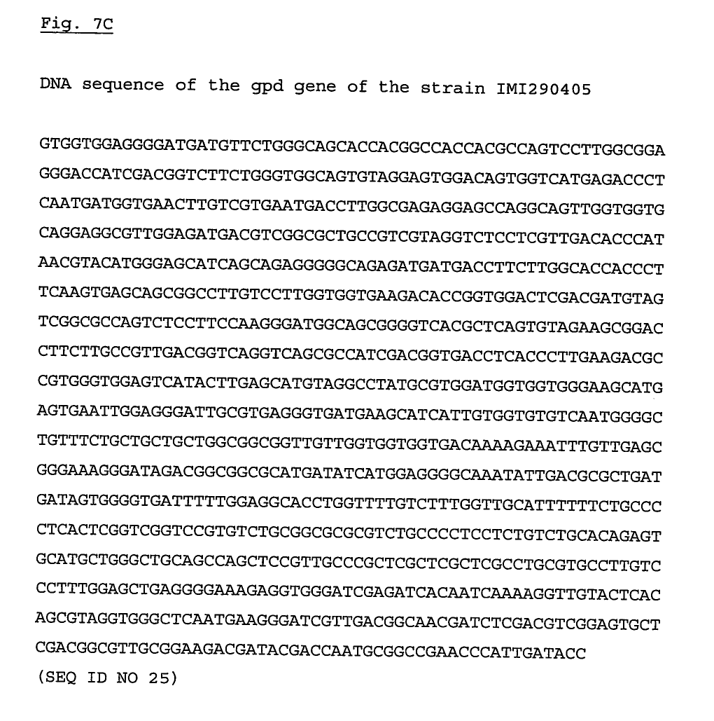

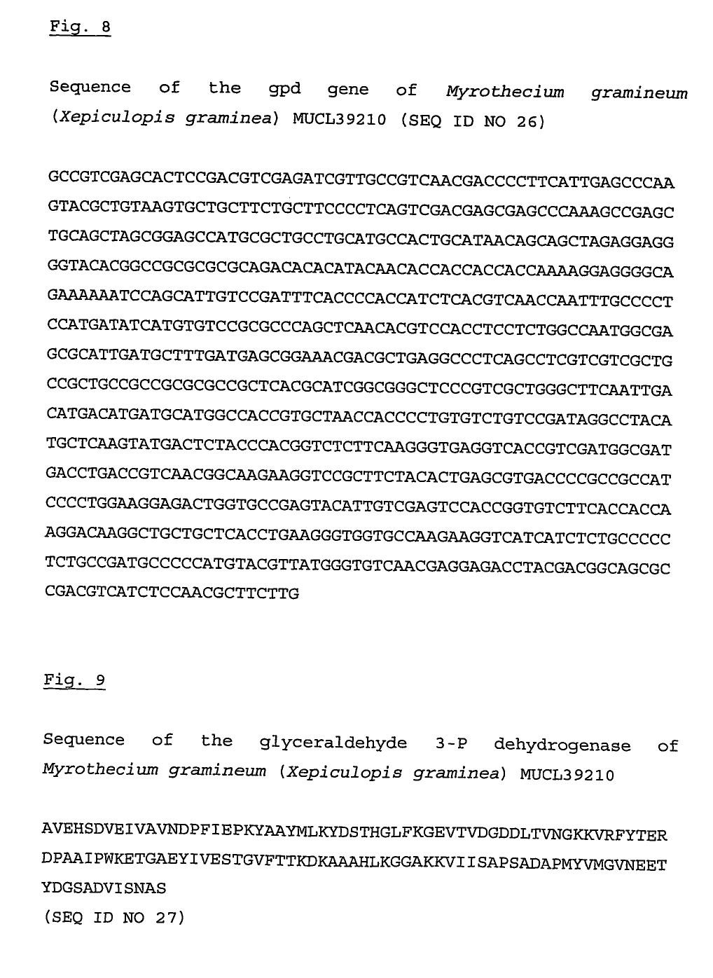

The results of the analysis of 5 different transformants of Myrothecium gramineum (syn. Xepiculopsis graminea) MUCL39210 selected for the hygromycin resistance and amylase production are shown on FIG. 4. The agarose gel electrophoresis of the PCR products shows that all the five transformants studied have integrated both the selection marker gene (hph gene—c.a. 1000 bp DNA fragment—lanes 4, 6, 8, 10 & 12) and the reporter gene (α-amylase gene of Aspergillus oryzae—c.a. 1100 bp DNA fragment—lanes 5, 7, 9, 11 & 13). Negative controls are presented in the lanes 2 & 3 (respectively products of hygromycine and α-amylase genes PCR amplification). Lanes 14 and 15 are positive controls (PCR reactions performed respectively on the pCSN43 and p2G-S plasmids).

3.2. Evaluation of Recombinant Protein Production

3.2.a. Culture of the Transformants

Twelve transformants of Myrothecium gramineum (syn. Xepiculopsis graminea) MUCL39210 obtained by transformation with the plasmids pCSN43 and p2G-S as well as 49 transformants of Aspergillus niger MUCL28817 have been cultivated in AMM medium with 3% sucrose as carbon source in shaking incubators (140 rpm) at 25° C. The culture volume was 20 ml for a total erlenmeyer volume of 100 ml. After 4 days, the supernatants of the cultures were collected by centrifugation.

3.2.b. α-Amylase Activity Determination

The α-amylase activity was determined by a modification of the method included in the Sigma Amylase kit. The substrate was diluted in a 100 mM NaH₂PO₄ buffer pH 6.0. One amylase unit was defined as the amount of enzyme that gives an absorbance of 10D unit at 405 nm after 15 min reaction at 37° C. and when 120 μl of the reaction mix are red in a 96 holes microplate with the model 550 microplate reader (Bio-Rad)

3.2.c. Biomass Determination (Dry Matter Determination)

The amount of biomass in a culture was determined by filtering an aliquot of the culture through a Whatman no 540 filter and putting the filter in an oven at 105° C. for 48 hours.

3.2.d. Results

TABLE 1

| | |
|---|---|
| MUCL39210 | MUCL28817 |

| | U/ml | U/g biomass | U/ml | U/g biomass |
|---|---|---|---|---|
| WT | 1.33 | 92.5 | 0.45 | 27.5 |
| Tf. 1 | 5.75 | 428 | 20.18 | 1127.5 |
| Tf. 2 | 44.5 | 3888 | 1.225 | 75 |
| Tf. 3 | 18.5 | 1488 | 26.03 | 1355 |
| Tf. 4 | 11.9 | 933 | 21.53 | 1145 |
| Tf. 5 | 20.8 | 1675 | 25.13 | 1325 |
| Tf. 6 | 21.5 | 1683 | 5.925 | 345 |
| Tf. 7 | 32.5 | 2348 | 24.53 | 1270 |
| Tf. 8 | 34.3 | 2718 | 7.95 | 405 |
| Tf. 9 | 59.5 | 4035 | 5.55 | 307.5 |
| Tf. 10 | 32.5 | 2850 | 22.05 | 1122.5 |
| Tf. 11 | 18.4 | 1430 | 2.3 | 142.5 |
| Tf. 12 | 46 | 3680 | 9.85 | 497.5 |
| Tf. 13 | | | 20.3 | 1127.5 |
| Tf. 14 | | | 25.35 | 1302.5 |
| Tf. 15 | | | 2.025 | 110 |
| Tf. 16 | | | 2.1 | 122.5 |
| Tf. 17 | | | 5.35 | 302.5 |
| Tf. 18 | | | 21.63 | 1205 |
| Tf. 19 | | | 2.725 | 152.5 |
| Tf. 20 | | | 25.35 | 1282.5 |
| Tf. 21 | | | 20.38 | 1032.5 |
| Tf. 22 | | | 22.18 | 1027.5 |
| Tf. 23 | | | 1.95 | 120 |
| Tf. 24 | | | 11.05 | 577.5 |
| Tf. 25 | | | 2.95 | 177.5 |
| Tf. 26 | | | 13.2 | 667.5 |
| Tf. 27 | | | 0.825 | 52.5 |
| Tf. 28 | | | 9.675 | 540 |
| Tf. 29 | | | 28.65 | 1552.5 |
| Tf. 30 | | | 1.775 | 110 |
| Tf. 31 | | | 26.18 | 1422.5 |
| Tf. 32 | | | 2.925 | 200 |
| Tf. 33 | | | 3.9 | 225 |
| Tf. 34 | | | 1.55 | 90 |
| Tf. 35 | | | 1.2 | 82.5 |
| Tf. 36 | | | 2.275 | 157.5 |
| Tf. 37 | | | 9.425 | 560 |
| Tf. 38 | | | 1.075 | 62.5 |
| Tf. 39 | | | 4.275 | 277.5 |
| Tf. 40 | | | 25.05 | 1370 |
| Tf. 41 | | | 8.175 | 462.5 |
| Tf. 42 | | | 1.7 | 110 |
| Tf. 43 | | | 1.075 | 70 |
| Tf. 44 | | | 1.15 | 70 |
| Tf. 45 | | | 2.275 | 147.5 |
| Tf. 46 | | | 1.525 | 105 |
| Tf. 47 | | | 1.475 | 90 |
| Tf. 48 | | | 27.75 | 1420 |
| Tf. 49 | | | 0.75 | 47.5 |
| Average | 28.8 | 2263 | 10.48 | 562.5 |

The table 1 shows that all transformants (Tf.) obtained with the strain Myrothecium gramineum (syn. Xepiculopsis graminea) MUCL39210 produce significantly more α-amylase than a non-transformed control strain. Many of the Aspergillus niger MUCL28817 transformants produce very few α-amylase compared to the non-transformed strain indicating that in those cases, they should not have been transformed by the reported gene or that this reporter gene is integrated under a non-expressible form.

The Myrothecium transformants produce more α-amylase than the Aspergillus transformants obtained and cultivated in the same conditions. The best Xepiculopsis transformant (no 9) produces more than twice α-amylase compared to the best Aspergillus transformant (no 29). On an average the Myrothecium transformants produce 2.75 more α-amylase than the Aspergillus transformants.

Furthermore, the Myrothecium transformants have a better specific production of α-amylase (amount of enzyme/g of biomass) compared to the Aspergillus transformants.

3.4. Stability of the Transformants

A hygromycin-resistant transformant of Myrothecium gramineum (syn. Xepiculopsis graminea) MUCL39210 was stored at −80° C. During the storage, the same transformant was successively replicated 20 times on solid PDA (Potato Dextrose Agar) medium without selective pressure. After these replica platings, the two strains (the original transformant and the replicated strain) together with the non-transformed Myrothecium gramineum (syn. Xepiculopsis graminae) MUCL39210 were grown (24° C.-145 rpm) in 20 ml of liquid medium (AMM medium supplemented with 3% sucrose and 0.5% yeast extract, pH 6.3) in 100 ml erlenmeyers. Samples of the cultures were taken at various times and assayed for α-amylase activity.

TABLE 2

Activity U/ml

| | 120 HF | 144 HF |
|---|---|---|
| Wild-type | 3.225 | 9.4 |
| Original transformant | 15.9 | 19.575 |
| Transformant replicated 20 times | 16.925 | 21.375 |

As shown in the table 2, the number of replications performed with the Myrothecium gramineum (syn. Xepiculopsis graminea) MUCL39210 transformant does not affect the productivity of the strain.

3.5. Analysis of Transformants of Other Myrothecium Strains

Similar results (enzymatic activity determination, control of the integration of the DNA constructs) as the ones obtained for transformants of Myrothecium gramineum (Xepiculopsis graminea) MUCL39210 have been obtained with transformants of other representative strains of the species Myrothecium gramineum as well as transformants of strains representative of the genus Myrothecium, i.e. Myrothecium gramineum IMI140595, Myrothecium gramineum CBS449.71, and Myrothecium verrucaria CBS328.52. According to the ribosomal DNA sequencing results, it appears that Myrothecium gramineum CBS449.71 could belong to another species than gramineum.

The same types of DNA constructs as for

Myrothecium gramineum (Xepiculopsis graminea) MUCL39210 have been used for the transformation of these strains. The transformants obtained have been evaluated as described in example 3.2.

The strains CBS449.71, IMI140595 and CBS328.52, and their transformants were grown (30° C.-145 rpm) in 70 ml of liquid medium (AMM medium supplemented with 3% sucrose and 0.5% yeast extract, pH 6.3) in 250 ml Erlenmeyer.

The amylase activity of the strains was measured after 123 hours fermentation. The results of amylase production are presented in table 3.

TABLE 3

| | | Relative |
|---|---|---|
| | | amylase |
| strain | | activity |
| CBS449.71 | wt. | 1 |
| CBS449.71 | transformant n°13 | 3.44 |
| CBS449.71 | transformant n°2 | 4.23 |
| CBS449.71 | transformant n°45 | 4.23 |
| CBS449.71 | transformant n°1 | 4.97 |
| IMI140595 | wt | 1 |
| IMI140595 | transformant n°22.1 | 1.86 |
| IMI140595 | transformant n°34.1 | 2.03 |
| IMI140595 | transformant n°1.1 | 13.81 |
| CBS328.52 | wt | 1 |
| CBS328.52 | transformant n°13 | 1.47 |
| CBS328.52 | transformant n°31 | 5.81 |
| CBS328.52 | transformant n°23 | 28.69 |

The applicant has made a deposit of some Myrothecium strains according to the invention at the BCCM/MUCL Culture Collection (Mycothèque de l'Université Catholique de Louvain, Place Croix du Sud 3, B-1348 LOUVAIN-LA-NEUVE, BELGIUM). Accession numbers are the following: MUCL44828 for the strain Myrothecium gramineum CBS449.71, MUCL44829 for the strain Myrothecium gramineum IMI140595, MUCL44830 for the strain Xepiculopsis graminea (Myrothecium gramineum) MUCL39210, MUCL44831 for the strain Myrothecium verrucaria CBS328.52 according to the invention. The above deposits under the Budapest Treaty according to Rule 28 EPC were made on 27 Aug. 2003.

1. A Myrothecium host cell comprising at least one recombinant DNA construct for modulated expression of homologous genes and/or for an expression of heterologous genes.

2. The host cell according to claim 1 wherein said recombinant DNA construct is integrated into a chromosome of the host cell and/or is present on an episome in the host cell.

3. The host cell according to claim 1 wherein said recombinant DNA construct comprises a nucleic acid sequence encoding a heterologous protein and/or a homologous protein.

4. The host cell according to claim 3 wherein said recombinant DNA construct comprises a nucleic acid sequence encoding a fungal protein.

5. The host cell according to claim 1 wherein said DNA construct comprises at least one homologous or heterologous tool that allows or enhances protein expression, said tool being selected from the group consisting of a promoter sequence, a terminator sequence, a polyadenylation signal sequence, a leader sequence, a secretion signal sequence, a selection marker gene sequence and reporter gene sequence.

6. The host cell according to claim 5 wherein said homologous or heterologous tool is a selection marker gene sequence is selected from the group consisting of a hygromycine B resistance gene sequence, phleomycin resistance gene sequence, a phosphinothricine resistance gene sequence, acetamidase gene sequence, a pyrG gene sequence, an argB gene sequence, a niaD gene sequence and a trpC gene sequence.

7. The host cell according to claim 5 wherein said homologous or heterologous tool is a promoter sequence is selected from the group consisting of an Aspergillus oryzae TAKA-amylase promoter sequence, an Rhizomucor miehei aspartic proteinase promoter sequence, an A. niger gluocamylase promoter sequence, an A. niger neutral α-amylase promoter sequence, an A. niger acid stable α-amylase promoter sequence, an R. miehei lipase promoter sequence and a promoter sequence of the glycolytic enzymes genes GPD, PGK and ADH.

8. The host cell according to claim 5 wherein said promoter is a regulatable promoter.

9. The host cell according to claim 1 selected from the group of Myrothecium sp. cells consisting of Myrothecium inundatum, Myrothecium prestonii, Myrothecium leucotrichum, Myrothecium cinctum, Myrothecium masonii, Myrothecium roridum, Myrothecium verrucaria, Myrothecium carmichaelii, Myrothecium lachastrae, Myrothecium atrum, Myrothecium atroviride, Myrothecium gramineum (syn. Xepiculopsis graminae) cells.

10. The host cell according to claim 9 wherein said host cell is selected from the group consisting of the Myrothecium gramineum strain MUCL39210, the Myrothecium gramineum strain CBS449.71, the Myrothecium gramineum IMI140595, the Myrothecium gramineum IMI290405 and the Myrothecium verrucaria strain CBS328 cells.

11. The host cells according to claim 9 wherein said host cell is the Myrothecium gramineum strain MUCL39210 cell.

12. The host cell according to claim 1 comprising a PCNS43 or a p3SR2 vector.

13. (canceled)

14. (canceled)

15. (canceled)

16. (canceled)

17. (canceled)

18. A method of genetically modifying Myrothecium sp. cells to generate Myrothecium host cells of claim 1, said method comprising the steps of:
growing Myrothecium cells or Myrothecium protoplasts;
by introducing into said Myrothecium cells or Myrothecium protoplasts at least one recombinant DNA construct for a modulated expression of homologous genes and/or for an expression of heterologous genes; and
selecting genetically modified Myrothecium cells or genetically modified Myrothecium protoplasts.

19. The method according to claim 18 wherein said recombinant DNA construct is a plasmid or a vector.

20. A genetically modified Myrothecium cell obtainable by a method according to claim 18.

21. The genetically modified cell according to claim 20 having an increased activity selected from the group consisting of increased amylase activity, increased xylanase activity, increased growth rate, and increased biomass production and/or having a reduced protease production.

22. The genetically modified cell according to claim 20 with an altered metabolic pathway compared to a nontransformed Myrothecium cell.

23. A method for producing a protein of interest, said method comprising the steps of:
culturing Myrothecium host cells of claim 1 under conditions which permit expression of the protein; and
recovering the protein from said Myrothecium cultured host cells.

24. The method according to claim 23 wherein said protein is a fungal protein.

25. The method according to claim 23 wherein said protein is selected from the group consisting of an enzyme, a therapeutic drug or a biopesticide.
